(19) United States
(12) Patent Application Publication
(10) Pub. No.: US 2024/0139037 A1
(43) Pub. Date: May 2, 2024

(54) ARRAY OF ABSORBENT ARTICLES WITH ULTRASONICALLY BONDED STRETCH LAMINATES

(71) Applicant: The Procter & Gamble Company, Cincinnati, OH (US)

(72) Inventor: Astrid Annette SHEEHAN, Symmes Township, OH (US)

(21) Appl. No.: 18/498,341

(22) Filed: Oct. 31, 2023

Related U.S. Application Data
(60) Provisional application No. 63/421,278, filed on Nov. 1, 2022.
(60) Provisional application No. 63/444,587, filed on Feb. 10, 2023.

(51) Int. Cl. A61F 13/49

(52) U.S. Cl. CPC A61F 13/4902; A61F 13/49011; A61F 2013/15869

(57) ABSTRACT

An array including at least two absorbent articles produced by the same manufacturer and/or sold under the same brand name is provided. Each absorbent article has side members made with an ultrasonically bonded laminate. The ultrasonically bonded laminates in the absorbent articles are different, and thereby are distinguishable, by two or more features selected from: ultrasonic bond shape, ultrasonic bond pattern, presence of frangible bonds, elastic film basis weight, first nonwoven primary bond pattern, and first nonwoven basis weight. At least one dimension of the first absorbent article may also be different than at least one dimension of the second absorbent article.

### CROSS REFERENCE TO RELATED APPLICATION

This application claims the benefit, under 35 U.S.C. § 119(e) of U.S. Provisional Application No. 63/421,278, filed on Nov. 1, 2022 and U.S. Provisional Application No. 63/444,587, filed on Feb. 10, 2023, the entire disclosures of each of which are fully incorporated by reference herein.

### FIELD

The present disclosure relates generally to arrays of absorbent articles and more specifically to arrays of absorbent articles comprising ultrasonically bonded stretch laminates.

### BACKGROUND

Manufacturers of disposable absorbent articles invest in multiple brands, tiers, and size lineups despite the added complexity. They do so to better meet the broad and ever evolving range of consumer aspirations over the lifecycle of their babies along with affordable economics. It therefore becomes important to offer product features that enable differentiation across sizes and tiers both for overt functional benefit as well as noticeable cues that signal underlying performance differences. Consumers, however, have always been known to multitask, and in modern times, they are noticed to be even less attentive and less sensitive to differences even with ordinary household products with which they interact regularly. This makes it essential for manufacturers to increase their investment in noticeability.

One of the key product features of disposable absorbent articles, such as diapers and pants, is the fundamental need for comfort and fit. The latter is judged at both initial application as well as during wear time and is normally achieved through product features that are extensible (i.e., stretch). One way in which manufacturers attempt to balance the competing interests of proper fit and variation in body type is through the use of expandable materials. One such group of materials is known as stretch laminates. As the name suggests, these materials are actually composites of individual components that are laminated or bonded together,

In recent years, commercialization of ultrasonically bonded laminates has gained favor by the absorbent article industry. These stretch engines or members are simpler in construction and favorable to cost and complexity because they can eliminate the need for adhesives, which requires hardware, frequent cleaning, and contributes to ongoing cost and product malodor. Importantly, these simpler stretch engines are subject to the same distinctiveness requirements as any other product function or component.

The ultrasonically bonded laminates may be used as front or back ears in taped diapers or as front or back side panels in pants, for example. The front or back ears and side panels require different characteristics to fit across brands, tiers and size lineups. As such there is a need to improve design flexibility to support brands, tiers, and size lineups without incurring complexity that offsets the minimalistic nature of ultrasonic laminate systems.

The discussion of shortcomings and needs existing in the field prior to the present disclosure is in no way an admission that such shortcomings and needs were recognized by those skilled in the art prior to the present disclosure.

### SUMMARY

Various embodiments solve the above-mentioned problems and provide arrays of absorbent articles that are useful for distinguishing absorbent articles produced by the same manufacturer and/or sold under the same brand name. Arrays of products that contain ultrasonically bonded laminates may be created, wherein the laminates differ by at least two or more specific variables. Contrary to expectations, it has unexpectedly been discovered that changing one variable alone may not produce meaningful distinction to the consumer. At times, one variable may produce a difference that is only noticeable in directed side-by-side comparisons, but this is insufficient for strong brand and tier positioning. A broad range of changes are conceivable, but the added complexity offsets the benefits of having a simple ultrasonic laminate construction in the first place.

Therefore, arrays according to this disclosure maximize design flexibility for best-in-class fit and comfort that is distinctive to consumers while avoiding added complexity. Such an array may comprise a first absorbent article comprising a first elastic side member and a second absorbent article comprising a second elastic side member. Each of the first and second elastic side members may comprise a first nonwoven material, an elastic film in a face-to-face relationship with the first nonwoven material, and a plurality of ultrasonic bonds joining the first nonwoven material and the elastic film to form an ultrasonically bonded laminate. The ultrasonically bonded laminate in the first absorbent article may be different than, and thereby distinguishable from, the ultrasonically bonded laminate in the second absorbent article by two or more features selected from: ultrasonic bond shape, ultrasonic bond pattern, presence of frangible bonds, elastic film basis weight, first nonwoven primary bond pattern, and first nonwoven basis weight. At least one dimension of the first absorbent article may also be different than at least one dimension of the second absorbent article.

These and other features, aspects, and advantages of various embodiments will become better understood with reference to the following description, figures, and claims.

### BRIEF DESCRIPTION OF THE DRAWINGS

Many aspects of this disclosure can be better understood with reference to the following figures.

FIG. 1 is a plan view of an example absorbent article in the form of a taped diaper, garment-facing surface facing the viewer, in a flat laid-out state.

FIG. 2 is a plan view of the example absorbent article of FIG. 1, wearer-facing surface facing the viewer, in a flat laid-out state.

FIG. 3 is a front perspective view of the absorbent article of FIGS. 1 and 2 in a fastened position.

FIG. 4 is a front perspective view of an absorbent article in the form of a pant.

FIG. 5 is a rear perspective view of the absorbent article of FIG. 4.

FIG. 6 is a plan view of the absorbent article of FIG. 4, laid flat, with a garment-facing surface facing the viewer.

FIG. 7 is a cross-sectional view of the absorbent article taken about line 7-7 of FIG. 6.

FIG. 8 is a cross-sectional view of the absorbent article taken about line 8-8 of FIG. 6.

FIG. 9 is a plan view of an example absorbent core or an absorbent article.

FIG. 10 is a cross-sectional view, taken about line 10-10, of the absorbent core of FIG. 9.

FIG. 11 is a cross-sectional view, taken about line 11-11, of the absorbent core of FIG. 9. FIGS. 12A-12F are a cross-sectional views of various stretch laminates.

FIG. 13A is a SEM photomicrograph showing a cross-sectional view of a portion of an elastomeric film that has not been pre-activated.

FIG. 13B is a magnified version of the SEM photomicrograph of FIG. 13A.

FIG. 14A is a SEM photomicrograph showing a cross-sectional view of a portion of a pre-activated elastomeric film.

FIG. 14B is a magnified version of the SEM photomicrograph of FIG. 14A.

FIG. 15 is a transmitted light photomicrograph of a top view of a portion of an elastomeric film that has not been pre-activated.

FIG. 16 is a transmitted light photomicrograph of a top view of a portion of a pre-activated elastomeric film illustrating activation stripes.

FIG. 17 is a schematic illustration of a continuous process for making a stretch laminate according to the present disclosure.

FIG. 18 is a schematic illustration of a plurality of individual ultrasonic bonds shapes.

FIG. 19 is a schematic illustration of an open cell bond pattern.

FIGS. 20A and 20B are schematic representations of exemplary closed cell bond patterns.

FIGS. 21A and 21B are plan views of an exemplary side members comprising laminates having more than one bond pattern.

FIGS. 22A and 22B are schematic illustrations of laminates having different bond densities

FIG. 23 is a schematic illustration of a stretch laminate being subjected to a lateral pull force.

FIGS. 24A-24C is a schematic illustration of a stretch laminate with various types of frangible bond sites after being subjected to a lateral pull force.

FIG. 25A-25F are schematic illustrations showing a variety of primary bond patterns for the nonwoven materials that may be employed according to various embodiments.

FIG. 26A is an exploded perspective view of an exemplary side member schematically illustrating exemplary surface modifications.

FIG. 26B is a plan view of an exemplary side member illustrating exemplary structural features.

FIGS. 27A-27D are schematic illustrations showing some exemplary arrays of absorbent articles.

FIG. 28 is a schematic illustration showing an exemplary back ear of an absorbent article, identifying features relevant for a back ear subject to the Laminate Extension Test.

FIG. 28A is a schematic illustration of a side view of an exemplary back ear identifying features relevant for a back ear subject to the Tensile Test Method.

FIGS. 29A-29B are schematic illustrations of an exemplary side member of an absorbent article and identifying features relevant for a side member subject to the Laminate Extension Test and the Tensile Test Method.

It should be understood that the various embodiments are not limited to the examples illustrated in the figures.

### DETAILED DESCRIPTION

### Introduction and Definitions

This disclosure is written to describe the invention to a person having ordinary skill in the art, who will understand that this disclosure is not limited to the specific examples or embodiments described. The examples and embodiments are single instances of the invention which will make a much larger scope apparent to the person having ordinary skill in the art. Unless defined otherwise, all technical and scientific terms used herein have the same meaning as commonly understood by the person having ordinary skill in the art. It is also to be understood that the terminology used herein is for the purpose of describing examples and embodiments only, and is not intended to be limiting, since the scope of the present disclosure will be limited only by the appended claims.

All the features disclosed in this specification (including any accompanying claims, abstract, and drawings) may be replaced by alternative features serving the same, equivalent, or similar purpose, unless expressly stated otherwise. Thus, unless expressly stated otherwise, each feature disclosed is one example only of a generic series of equivalent or similar features. The examples and embodiments described herein are for illustrative purposes only and that various modifications or changes in light thereof will be suggested to the person having ordinary skill in the art and are to be included within the spirit and purview of this application. Many variations and modifications may be made to the embodiments of the disclosure without departing substantially from the spirit and principles of the disclosure. All such modifications and variations are intended to be included herein within the scope of this disclosure. For example, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

All numeric values are herein assumed to be modified by the term “about,” whether or not explicitly indicated. The term “about” generally refers to a range of numbers that one of skill in the art would consider equivalent to the recited value (for example, having the same function or result). In many instances, the term “about” may include numbers that are rounded to the nearest significant figure.

In everyday usage, indefinite articles (like “a” or “an”) precede countable nouns and noncountable nouns almost never take indefinite articles. It must be noted, therefore, that, as used in this specification and in the claims that follow, the singular forms “a,” “an,” and “the” include plural referents unless the context clearly dictates otherwise. Thus, for example, reference to “a support” includes a plurality of supports. Particularly when a single countable noun is listed as an element in a claim, this specification will generally use a phrase such as “a single.” For example, “a single support.”

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

In this specification and in the claims that follow, reference will be made to a number of terms that shall be defined to have the following meanings unless a contrary intention is apparent.

“Absorbent article” refers to devices that absorb and contain liquid, and more specifically, refers to devices that are placed against or in proximity to the body of the wearer to absorb and to contain various exudates discharged from the body.

“Activated” or “pre-activated” refer to a process of mechanically deforming a material in order to increase the extensibility of at least a portion of the material. A material may be activated or pre-activated by, for example, incrementally stretching the material in at least one direction.

“Adhesively bonded” or “adhesively laminated” refer to a laminate wherein an adhesive is used to bond an elastomeric material to at least one cover layer.

“Attached” refers to elements being connected or united by fastening, adhering, bonding, or by any other method suitable for connecting the elements together and to their constituent materials. Many suitable methods for attaching elements together are well-known, including adhesive bonding, pressure bonding, thermal bonding, ultrasonic bonding, mechanical fastening, etc. Such attachment methods may be used to attach elements together over a particular area either continuously or intermittently.

“Diaper” refers to an absorbent article generally worn by infants and incontinent persons about the lower torso and having the general form of a sheet, different portions of which are fastened together to encircle the waist and the legs of the wearer.

“Disposable” refers to absorbent articles that generally are not intended to be laundered or otherwise restored or reused as absorbent articles, i.e., they are intended to be discarded after a single use and, preferably, to be recycled, composted or otherwise disposed of in an environmentally compatible manner.

“Disposed” is used to mean that an element(s) is formed (joined and positioned) in a particular place or position as a unitary structure with other elements or as a separate element joined to another element.

“Extensible” refers to the property of a material, wherein: when a biasing force is applied to the material, the material can be extended to an elongated length of at least 115% of its original relaxed length (i.e., can extend 15%), without a rupture or breakage that renders the material unusable for its intended purpose. A material that does not meet this definition is considered inextensible. In some embodiments, an extensible material may be able to be extended to an elongated length of 125% or more of its original relaxed length without rupture or breakage that renders the material unusable for its intended purpose. An extensible material may or may not exhibit recovery after application of a biasing force.

Throughout the present disclosure, an extensible material is considered to be “elastically extensible” if, when a biasing force is applied to the material, the material can be extended to an elongated length of at least 115% of its original relaxed length (i.e., can extend 15%), without rupture or breakage which renders the material unusable for its intended purpose, and when the force is removed from the material, the material recovers at least 40% of its elongation. In various examples, when the force is removed from an elastically extensible material, the material may recover at least 60%, or at least 80%, of its elongation.

An “elastic,” “elastomer” or “elastomeric” refers to materials exhibiting elastic properties, which include any material that upon application of a force to its relaxed, initial length can stretch or elongate to an elongated length more than 15% greater than its initial length and will substantially recover back to about its initial length upon release of the applied force.

“Interior” and “exterior” refer respectively to the location of an element that is intended to be placed against or toward the body of a wearer when an absorbent article is worn and the location of an element that is intended to be placed against or toward any clothing that is worn over the absorbent article. Synonyms for “interior” include, but are not limited to “inner,” “inside,” “skin-facing,” “skin-side,” “wearer-facing,” or “wearer-side.” Synonyms for “exterior include, but are not limited to “outer,” “outside,” “garment-side,” or “garment-facing.” Also, in the taped diaper context, when the absorbent article is oriented such that its interior faces upward, e.g., when it is laid out in preparation for setting the wearer on top of it, synonyms include “upper” and “lower” and “top” and “bottom”, respectively.

“Joined” refers to configurations whereby an element is directly secured to another element by attaching the element directly to the other element, and configurations whereby an element is indirectly secured to another element by attaching the element to intermediate member(s) which in turn are attached to the other element.

“Lateral” or “transverse” refers to a direction running at a 90 degree angle to the longitudinal direction and includes directions within ±45° of the lateral direction.

“Longitudinal” refers to a direction running parallel to the maximum linear dimension of the article and includes directions within ±45° of the longitudinal direction.

“Pant” or “pants” refers to an absorbent article generally worn by infants and incontinent persons about the lower torso and having the general form of a pair of short pants that can be applied or removed from the wearer without unfastening. A pant may be placed in position on the wearer by inserting the wearer's legs into the leg openings and sliding the pant into position about the wearer's lower torso. While the term “pant” is used herein, pants are also commonly referred to as “closed diapers”, “prefastened diapers”, “pull-on diapers”, “training pants” and “diaper-pants”.

“Recovery” refers to ability of a material to return to its original size after it has been stretched.

“Refastenable” refers to the property of two elements being capable of releasable attachment, separation, and subsequent releasable reattachment without substantial permanent deformation or rupture.

“Releasably attached,” “releasably engaged,” and variations thereof refer to two elements being connected or connectable such that the elements tend to remain connected absent a separation force applied to one or both of the elements, and the elements being capable of separation without substantial permanent deformation or rupture. The required separation force is typically beyond that encountered while wearing the absorbent garment.

“Strain” or “percent strain” of a material is calculated by subtracting the original length from the stretched length, then dividing the result by the original length and multiplying by 100. The percent strain is described by the equation below:

Percent Strain=% Strain=Strain=100*[(Ls−L₀)/L₀]

where L₀ is the original length of the stretch laminate (or elastomeric film) at the beginning of the stretch step, and Ls is the length of the stretched laminate (or elastomeric film) at the end of the stretch step. A sample stretched from an original length of 10 mm to a length of 30 mm results in a strain of 200%. Strain can be calculated in a length direction, a width direction, or any direction there between.

“Set” or “percent set” of a material is calculated by subtracting an original length from a final length, then dividing the result by the original length and multiplying by 100. The percent set is described by the equation below:

Percent Set=% Set=Set=100*[(L_{f}−L₀)/L₀]

where L₀ is an original length of the stretch laminate (or elastomeric film) at the beginning of the stretch step, and L_{f} is a length of the relaxed stretch laminate (or elastomeric film) after it is relaxed from the stretch step. A sample is stretched from an original length of 10 mm to a length of 30 mm. Upon relaxing (removal of stress), the sample returns to 15 mm. This results in a set of 50%. Set can be calculated in a length direction, a width direction, or any direction there between.

“Wrinkle” refers to a small fold, ridge or crease.

“Align” or “aligned” or “aligning” means to place or to arrange in a straight line. Aligning edges of substrates, therefore, means arranging the substrates so that the edges in question extend along approximately the same line. It is to be appreciated that aligning edges of substrates can be accomplished in a variety of ways, including placing the substrates one on top of the other or side by side.

“Facing relationship” refers to a relative positioning of materials, such as substrates, in which a surface of one material is oriented toward a surface of another material. For example, when two substrates are stacked on top of each other, they are in a facing relationship. The term does not require or exclude the presence of intervening objects, materials, or layers.

“Machine direction” (MD) refers to the direction of material flow through a process. In addition, relative placement and movement of material can be described as flowing in the machine direction through a process from upstream in the process to downstream in the process.

“Cross direction” (CD) refers to a direction that is generally perpendicular to the machine direction.

“Nonwoven” refers herein to a material made from continuous (long) filaments (fibers) and/or discontinuous (short) filaments (fibers) by processes such as spunbonding, meltblowing, carding, and the like. In some configurations, a nonwoven may comprise a polyolefin based nonwoven, including but not limited to nonwovens having polypropylene fibers and/or polyethylene fibers and/or bicomponent fibers comprising a polyolefin. Nonlimiting examples of suitable fibers include spunbond, spunlaid, meltblown, spunmelt, solvent-spun, electrospun, carded, film fibrillated, melt-film fibrillated, air-laid, dry-laid, wet-laid staple fibers, and other nonwoven web materials formed in part or in whole of polymer fibers as known in the art, and workable combinations thereof. Nonwovens do not have a woven or knitted filament pattern. It is to be appreciated that nonwovens having various basis weights can be used in accordance with the methods herein. For example, some nonwovens may have a basis weight of at least about 8 gsm, 12 gsm, 16 gsm, 20 gsm, 25 gsm, 30 gsm, 40 gsm, or 65 gsm. Some nonwovens may have basis weight of about 8 gsm to about 65 gsm, or about 10 to about 22 gsm, specifically reciting all 1 gsm increments within the above-recited ranges and all ranges formed therein or thereby. The nonwovens may comprise or be formed of natural fibers, such as cotton, and bio-based fibers, such as bio-PE or bio-PP.

“Pattern” as used herein means a decorative or distinctive design, not necessarily repeating or imitative, including but not limited to the following: clustered, geometric, spotted, helical, swirl, arrayed, textured, spiral, cycle, contoured, laced, tessellated, starburst, lobed, blocks, pleated, concave, convex, braided, tapered, and combinations thereof. In some embodiments, the pattern may comprise one or more repeating design elements.

### General Description of an Absorbent Article

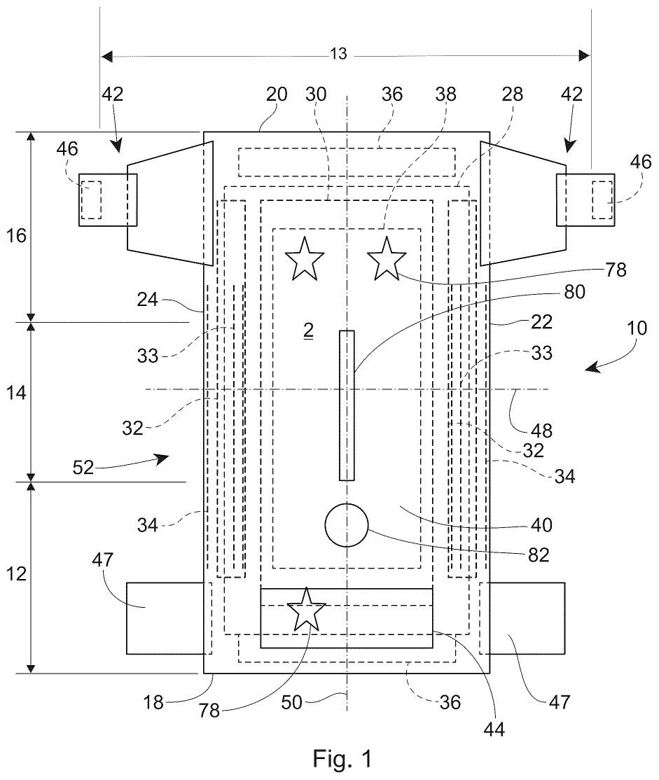

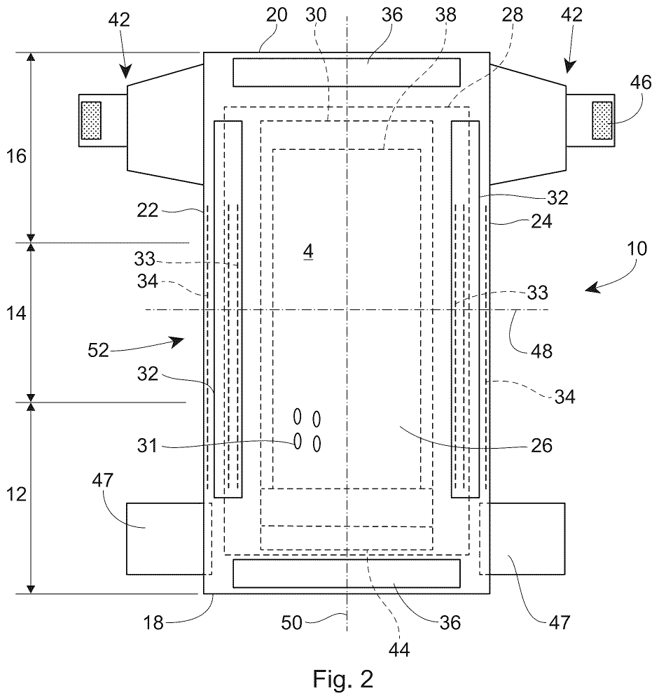

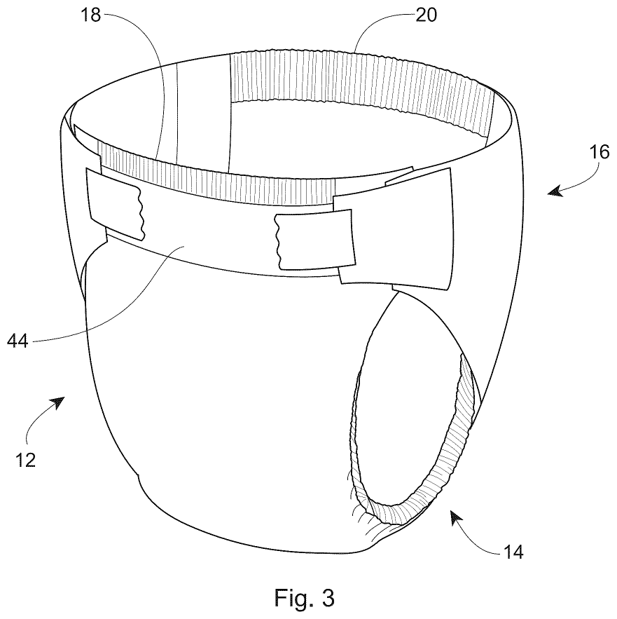

An example absorbent article 10 according to the present disclosure, shown in the form of a taped diaper, is represented in FIGS. 1-3. FIG. 1 is a plan view of the example absorbent article 10, garment-facing surface 2 facing the viewer in a flat, laid-out state (i.e., no elastic contraction). FIG. 2 is a plan view of the example absorbent article 10 of FIG. 1, wearer-facing surface 4 facing the viewer in a flat, laid-out state. FIG. 3 is a front perspective view of the absorbent article 10 of FIGS. 1 and 2 in a fastened configuration. The absorbent article 10 of FIGS. 1-3 is shown for illustration purposes only as the present disclosure may be used for making a wide variety of diapers, including adult incontinence products, pants, or other absorbent articles, such as sanitary napkins and absorbent pads, for example.

The absorbent article 10 may comprise a front waist region 12, a crotch region 14, and a back waist region 16. The crotch region 14 may extend intermediate the front waist region 12 and the back waist region 16. The front wait region 12, the crotch region 14, and the back waist region 16 may each be ⅓ of the length of the absorbent article 10. The absorbent article 10 may comprise a front end edge 18, a back end edge 20 opposite to the front end edge 18, and longitudinally extending, transversely opposed side edges 22 and 24 defined by the chassis 52.

The absorbent article 10 may comprise a liquid permeable topsheet 26, a liquid impermeable backsheet 28, and an absorbent core 30 positioned at least partially intermediate the topsheet 26 and the backsheet 28. The absorbent article 10 may also comprise one or more pairs of barrier leg cuffs 32 with or without elastics 33, one or more pairs of leg elastics 34, one or more elastic waistbands 36, and/or one or more acquisition materials 38. The acquisition material or materials 38 may be positioned intermediate the topsheet 26 and the absorbent core 30. A masking layer may be present intermediate the absorbent core and the backsheet film. An outer cover material 40, such as a nonwoven material, may cover a garment-facing side of the backsheet 28. The absorbent article 10 may comprise back ears 42 in the back waist region 16. The back ears 42 may comprise fasteners 46 and may extend from the back waist region 16 of the absorbent article 10 and attach (using the fasteners 46) to the landing zone area or landing zone material 44 on a garment-facing portion of the front waist region 12 of the absorbent article 10. The absorbent article 10 may also have front ears 47 in the front waist region 12. The absorbent article 10 may have a central lateral (or transverse) axis 48 and a central longitudinal axis 50. The central lateral axis 48 extends perpendicular to the central longitudinal axis 50. The absorbent article may comprise a secondary fastening system in addition to the primary fastening systems in a typed diaper context.

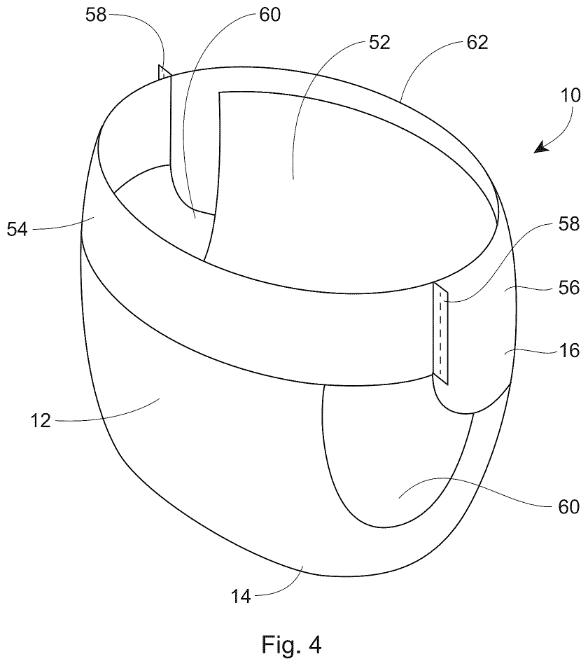

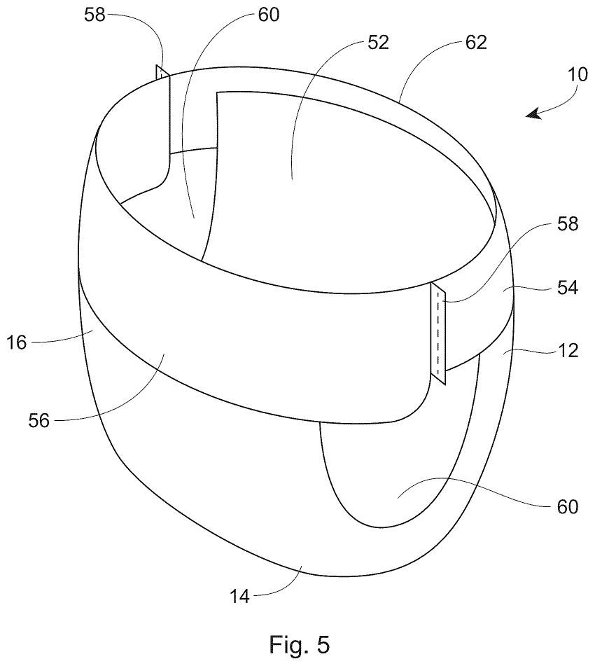

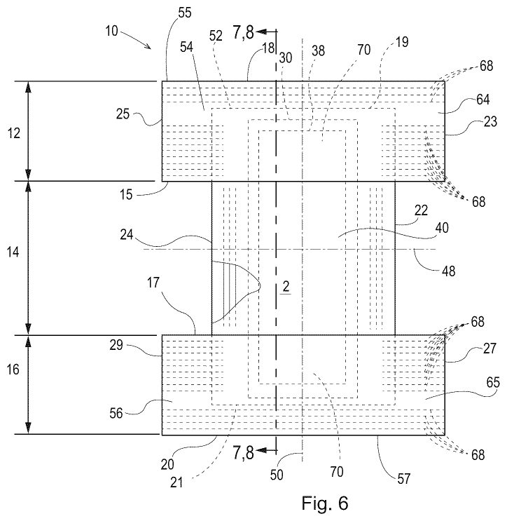

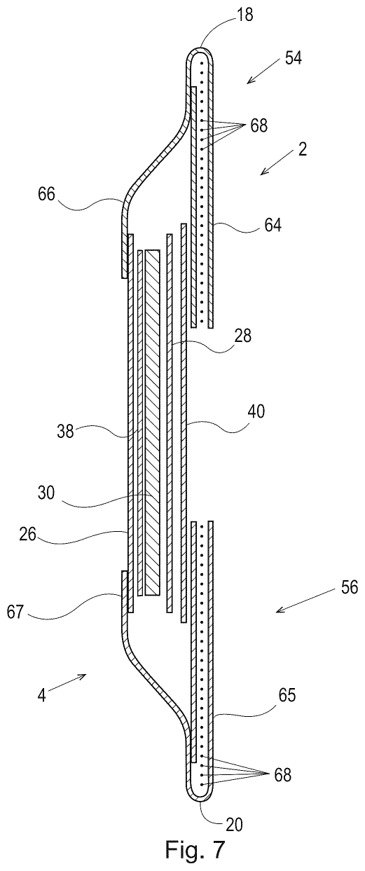

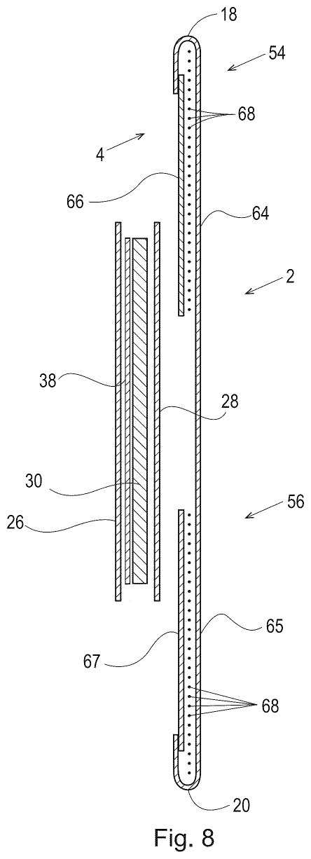

In other instances, the absorbent article may be in the form of a pant having permanent or refastenable side seams. Suitable refastenable seams are disclosed in U.S. Pat. Appl. Pub. No. 2014/0005020 and U.S. Pat. No. 9,421,137. Referring to FIGS. 4-8, an example absorbent article 10 in the form of a pant is illustrated. FIG. 4 is a front perspective view of the absorbent article 10. FIG. 5 is a rear perspective view of the absorbent article 10. FIG. 6 is a plan view of the absorbent article 10, laid flat, with the garment-facing surface facing the viewer. Elements of FIG. 4-8 having the same reference number as described above with respect to FIGS. 1-3 may be the same element (e.g., absorbent core 30). FIG. 7 is an example cross-sectional view of the absorbent article taken about line 7-7 of FIG. 6. FIG. 8 is an example cross-sectional view of the absorbent article taken about line 8-8 of FIG. 6. FIGS. 7 and 8 illustrate example forms of front and back belts 54, 56. The absorbent article 10 may have a front waist region 12, a crotch region 14, and a back waist region 16. Each of the regions 12, 14, and 16 may be ⅓ of the length of the absorbent article 10. The absorbent article 10 may have a chassis 52 (sometimes referred to as a central chassis or central panel) comprising a topsheet 26, a backsheet 28, and an absorbent core 30 disposed at least partially intermediate the topsheet 26 and the backsheet 28, and an optional acquisition material 38, similar to that as described above with respect to FIGS. 1-3. The absorbent article 10 may comprise a front belt 54 in the front waist region 12 and a back belt 56 in the back waist region 16. The chassis 52 may be joined to a wearer-facing surface 4 of the front and back belts 54, 56 or to a garment-facing surface 2 of the belts 54, 56. Side edges 23 and 25 of the front belt 54 may be joined to side edges 27 and 29, respectively, of the back belt 56 to form two side seams 58. The side seams 58 may be any suitable seams known to those of skill in the art, such as butt seams or overlap seams, for example. When the side seams 58 are permanently formed or refastenably closed, the absorbent article 10 in the form of a pant has two leg openings 60 and a waist opening circumference 62. The side seams 58 may be permanently joined using adhesives or bonds, for example, or may be refastenably closed using hook and loop fasteners, for example.

### Belts

Referring to FIGS. 7 and 8, the front and back belts 54 and 56 may comprise front and back inner belt layers 66 and 67 and front and back outer belt layers 64 and 65 having an elastomeric material (e.g., strands 68 or a film (which may be apertured)) disposed at least partially therebetween. The elastic elements 68 or the film may be relaxed (including being cut) to reduce elastic strain over the absorbent core 30 or, may alternatively, run continuously across the absorbent core 30. The elastics elements 68 may have uniform or variable spacing therebetween in any portion of the belts. The elastic elements 68 may also be pre-strained the same amount or different amounts. The front and/or back belts 54 and 56 may have one or more elastic element free zones 70 where the chassis 52 overlaps the belts 54, 56. In other instances, at least some of the elastic elements 68 may extend continuously across the chassis 52.

The front and back inner belt layers 66, 67 and the front and back outer belt layers 64, 65 may be joined using adhesives, heat bonds, pressure bonds, thermoplastic bonds, or ultrasonic bonds. Various suitable belt layer configurations can be found in U.S. Pat. Appl. Pub. No. 2013/0211363. Any of the belts 54 and 56 may comprise a stretch laminate as described hereinafter.

Front and back belt end edges 55 and 57 may extend longitudinally beyond the front and back chassis end edges 19 and 21 (as shown in FIG. 6) or they may be co-terminus. The front and back belt side edges 23, 25, 27, and 29 may extend laterally beyond the chassis side edges 22 and 24. The front and back belts 54 and 56 may be continuous (i.e., having at least one layer that is continuous) from belt side edge to belt side edge (e.g., the transverse distances from 23 to 25 and from 27 to 29). Alternatively, the front and back belts 54 and 56 may be discontinuous from belt side edge to belt side edge (e.g., the transverse distances from 23 to 25 and 27 to 29), such that they are discrete.

As disclosed in U.S. Pat. No. 7,901,393, the longitudinal length (along the central longitudinal axis 50) of the back belt 56 may be greater than the longitudinal length of the front belt 54, and this may be particularly useful for increased buttocks coverage when the back belt 56 has a greater longitudinal length versus the front belt 54 adjacent to or immediately adjacent to the side seams 58.

The front outer belt layer 64 and the back outer belt layer 65 may be separated from each other, such that the layers are discrete or, alternatively, these layers may be continuous, such that a layer runs continuously from the front belt end edge 55 to the back belt end edge 57. This may also be true for the front and back inner belt layers 66 and 67—that is, they may also be longitudinally discrete or continuous. Further, the front and back outer belt layers 64 and 65 may be longitudinally continuous while the front and back inner belt layers 66 and 67 are longitudinally discrete, such that a gap is formed between them—a gap between the front and back inner and outer belt layers 64, 65, 66, and 67 is shown in FIG. 7 and a gap between the front and back inner belt layers 66 and 67 is shown in FIG. 8.

The front and back belts 54 and 56 may include slits, holes, and/or perforations providing increased breathability, softness, and a garment-like texture. Underwear-like appearance can be enhanced by substantially aligning the waist and leg edges at the side seams 58 (see FIGS. 4 and 5).

The front and back belts 54 and 56 may comprise graphics (see e.g., 78 of FIG. 1). The graphics may extend substantially around the entire circumference of the absorbent article 10 and may be disposed across side seams 58 and/or across proximal front and back belt seams 15 and 17; or, alternatively, adjacent to the seams 58, 15, and 17 in the manner described in U.S. Pat. No. 9,498,389 to create a more underwear-like article. The graphics may also be discontinuous.

Alternatively, instead of attaching belts 54 and 56 to the chassis 52 to form a pant, discrete side panels may be attached to side edges of the chassis 22 and 24. Suitable forms of pants comprising discrete side panels are disclosed in U.S. Pat. Nos. 6,645,190; 8,747,379; 8,372,052; 8,361,048; 6,761,711; 6,817,994; 8,007,485; 7,862,550; 6,969,377; 7,497,851; 6,849,067; 6,893,426; 6,953,452; 6,840,928; 8,579,876; 7,682,349; 7,156,833; and 7,201,744.

### Topsheet

The topsheet 26 is the part of the absorbent article 10 that is in contact with the wearer's skin. The topsheet 26 may be joined to portions of the backsheet 28, the absorbent core 30, the barrier leg cuffs 32, and/or any other layers as is known to those of ordinary skill in the art. The topsheet 26 may be compliant, soft-feeling, and non-irritating to the wearer's skin. Further, at least a portion of, or all of, the topsheet may be liquid permeable, permitting liquid bodily exudates to readily penetrate through its thickness. The topsheet may be formed of one or more layers of equal or unequal size or area. A suitable topsheet may be manufactured from a wide range of materials, such as porous foams, reticulated foams, apertured plastic films, woven materials, nonwoven materials, woven or nonwoven materials of natural fibers (e.g., wood or cotton fibers), synthetic fibers or filaments (e.g., polyester or polypropylene or bicomponent PE/PP fibers or bio-sourced fibers, bio-polymers, PIR polymers or mixtures thereof), or a combination of natural and synthetic fibers. The topsheet may have one or more layers. The topsheet may be apertured (FIG. 2, element 31), may have any suitable three-dimensional features, and/or may have a plurality of embossments (e.g., a bond pattern). The topsheet may be apertured by overbonding a material and then rupturing the overbonds through ring rolling, such as disclosed in U.S. Patent No. 5,628,097, to Benson et al., issued on May 13, 1997 and disclosed in U.S. Pat. Appl. Publication No. US 2016/0136014 to Arora et al. Any portion of the topsheet may be coated with a skin care composition, an antibacterial agent, a surfactant, and/or other beneficial agents. The topsheet may be hydrophilic or hydrophobic or may have hydrophilic and/or hydrophobic portions or layers. If the topsheet is hydrophobic, typically apertures will be present so that bodily exudates may pass through the topsheet.

### Backsheet

The backsheet 28 is generally that portion of the absorbent article 10 positioned proximate to the garment-facing surface of the absorbent core 30. The backsheet 28 may be joined to portions of the topsheet 26, the outer cover material 40, the absorbent core 30, and/or any other layers of the absorbent article by any attachment methods known to those of skill in the art. The backsheet 28 prevents, or at least inhibits, the bodily exudates absorbed and contained in the absorbent core 10 from soiling articles such as bedsheets, undergarments, and/or clothing. The backsheet is typically liquid impermeable, or at least substantially liquid impermeable. The backsheet may, for example, be or comprise a thin plastic film, such as a thermoplastic film having a thickness of about 0.012 mm to about 0.051 mm. Other suitable backsheet materials may include breathable materials which permit vapors to escape from the absorbent article, while still preventing, or at least inhibiting, bodily exudates from passing through the backsheet. The backsheet may be printed with inks for graphics. The backsheet may also be printed with a wetness indicator, such as through the use of one or more hydrochromic inks.

### Outer Cover Material

The outer cover material (sometimes referred to as a backsheet nonwoven) 40 may comprise one or more nonwoven materials joined to the backsheet 28 and that covers the backsheet 28. The outer cover material 40 forms at least a portion of the garment-facing surface 2 of the absorbent article 10 and effectively “covers” the backsheet 28 so that film is not present on the garment-facing surface 2. The outer cover material 40 may comprise a bond pattern, apertures, and/or three-dimensional features. The outer cover material 40 may be a hydroentangled nonwoven material.

### Absorbent Core

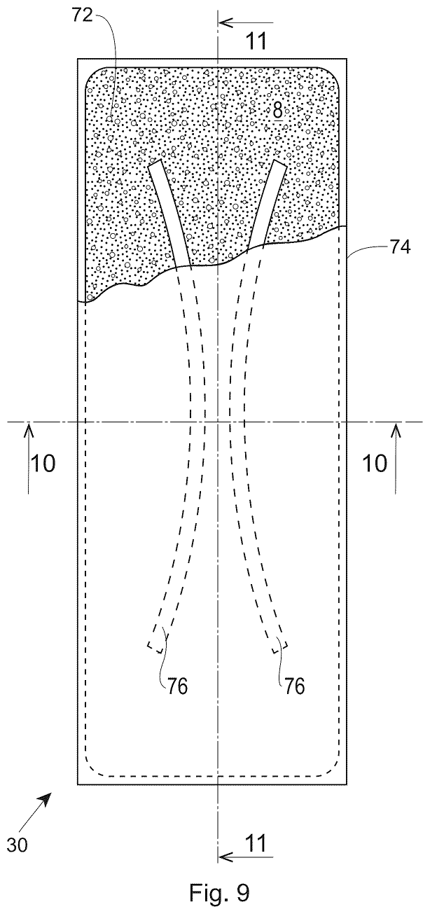

As used herein, the term “absorbent core” 30 refers to a component of the absorbent article 10 disposed in the article for absorbing and containing liquid such as urine received by the absorbent article. The absorbent core thus typically has a high absorbent capacity. An example absorbent core 30 is schematically shown in FIGS. 9-11. The absorbent core comprises an absorbent material 72, that is typically enclosed within or sandwiched between a core bag 74.

The core wrap may be a single material that is folded and attached to itself, or it may comprise a separate top layer and bottom layer that may be bonded or otherwise joined together. The absorbent material typically comprises superabsorbent particles which are optionally mixed with cellulose fibers. As used herein, “absorbent core” does not include any acquisition-distribution systems, topsheet, or backsheet of the absorbent article.

The example absorbent core 30 shown in isolation in FIGS. 9-11 is in the dry state (before use). The absorbent core may typically have a generally rectangular shape as defined by its longitudinal edges and transversal front edge and back edge or may have other shapes.

Absorbent material 72 may be deposited as an absorbent layer having a generally rectangular outline, as represented in FIG. 9. A wide variety of absorbent cores may also be used. The absorbent material 72 layer may also have a non-rectangular perimeter (“shaped” core), in particular, the absorbent material 72 may define a tapering along its width towards the central region of the core (or “dog-bone” shape). In this way, the absorbent material deposition area may have a relatively narrow width in an area of the core intended to be placed in the crotch region or towards the front region of the absorbent article. This may provide for example better wearing comfort. Other shapes can also be used such as a “T” or “Y” or “hourglass” for the area of the absorbent material.

The absorbent material 72 may be any conventional absorbent material known in the art. For example, the absorbent material may comprise a blend of cellulose fibers and superabsorbent particles (“SAP”), typically with the percentage of SAP ranging from about 50% to about 75% by weight of the absorbent material. According to various embodiments, the absorbent material may comprise at least 80% superabsorbent polymers by weight of the absorbent material. The absorbent material may also be free of cellulose fibers, as is known in so-called airfelt-free cores, where the absorbent material consists, or consists essentially, of SAP. The absorbent material may also be a high internal phase emulsion foam

“Superabsorbent polymer” or “SAP” refers herein to absorbent materials, typically cross-linked polymeric materials, that can absorb at least 10 times their weight of an aqueous 0.9% saline solution as measured using the Centrifuge Retention Capacity (CRC) test (EDANA method WSP 241.2.R3 (12)). The SAP may in particular have a CRC value of at least 20 g/g, in particular of from 20 g/g to 40 g/g. “Superabsorbent polymer particles”, as used herein, refers to a superabsorbent polymer material which is in particulate form so as to be flowable in the dry state.

Various absorbent core designs comprising high amounts of SAP have been proposed in the past, see for example in U.S. Pat. No. 5,599,335 (Goldman), EP1,447,066 (Busam), WO95/11652 (Tanzer), U.S. Pat. Appl. Pub. No. 2008/0312622A1 (Hundorf), WO2012/052172 (Van Malderen). In particular, the SAP printing technology as disclosed in U.S. Pat. Appl. Pub. No. 2006/024433 (Blessing), U.S. Pat. Appl. Pub. No. 2008/0312617 and U.S. Pat. Appl. Pub. No. 2010/0051166A1 (both to Hundorf et al.) may be used. The present disclosure however is not limited to a particular type of absorbent core. The absorbent core may also comprise one or more glues such as an auxiliary glue applied between the internal surface of one (or both) of the core wrap layers and the absorbent material to reduce leakage of SAP outside the core wrap. A micro-fibrous adhesive net may also be used in air-felt free cores as described in the above Hundorf references. These glues are not represented in the Figures for simplicity. Other core constructions comprising a high loft nonwoven substrate such as a carded nonwoven layer, having a porous structure into which SAP particles have been deposited, may also be used in present disclosure.

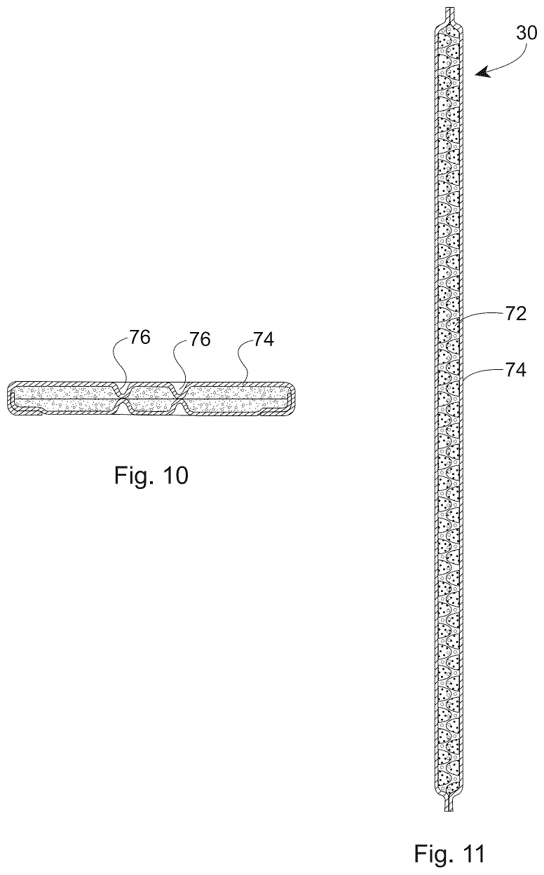

The absorbent material may be deposited as a continuous layer within the core wrap. The absorbent material may also be present discontinuously, for example, as individual pockets or stripes of absorbent material enclosed within the core wrap and separated from each other by material-free junction areas. A continuous layer of absorbent material, in particular of SAP, may also be obtained by combining two absorbent layers having matching discontinuous absorbent material application pattern, wherein the resulting layer is substantially continuously distributed across the absorbent particulate polymer material area, as illustrated in FIGS. 10-11. As for example taught in U.S. Pat. Appl. Pub. No. 2008/312,622A1 (Hundorf), each absorbent material layer may thus comprise a pattern having absorbent material land areas and absorbent material-free junction areas, wherein the absorbent material land areas of the first layer correspond substantially to the absorbent material-free junction areas of the second layer and vice versa.

The basis weight (amount deposited per unit of surface) of the absorbent material may also be varied to create a profiled distribution of absorbent material, in particular in the longitudinal direction to provide more absorbency towards the center and the middle of the core, but also in the transversal direction, or both directions of the core. The absorbent core may also comprise one or more longitudinally (or otherwise) extending channels 76, which are areas of the absorbent layer substantially free of absorbent material within the absorbent material layer. The top side of the core wrap may be advantageously bonded to the bottom side of the core by adhesive, mechanical or ultra-sonic bonding through these material-free areas. Example disclosures of such channels in an airfelt-free core can be found in WO2012/170778 (Rosati et al.) and US2012/0312491 (Jackels). Channels may of course also be formed in absorbent cores comprising a mix of cellulose fibers and SAP particles. These channels may embody any suitable shapes and any suitable number of channels may be provided. In other instances, the absorbent core may be embossed to create the impression of channels. The absorbent core in FIGS. 9-11 is merely an example absorbent core. Many other absorbent cores with or without channels are also within the scope of the present disclosure.

### Barrier Leg Cuffs/Leg Elastics

Referring to FIGS. 1 and 2, for example, the absorbent article 10 may comprise one or more pairs of barrier leg cuffs 32 and one or more pairs of leg elastics 34. The barrier leg cuffs 32 may be positioned laterally inboard of leg elastics 34. Each barrier leg cuff 32 may be formed by a piece of material which is bonded to the absorbent article 10 so it can extend upwards from a wearer-facing surface 4 of the absorbent article 10 and provide improved containment of body exudates approximately at the junction of the torso and legs of the wearer. The barrier leg cuffs 32 are delimited by a proximal edge joined directly or indirectly to the topsheet and/or the backsheet and a free terminal edge, which is intended to contact and form a seal with the wearer's skin. The barrier leg cuffs 32 may extend at least partially between the front end edge 18 and the back end edge 20 of the absorbent article 10 on opposite sides of the central longitudinal axis 50 and may be at least present in the crotch region 14. The barrier leg cuffs 32 may each comprise one or more elastics 33 (e.g., elastic strands or strips) near or at the free terminal edge. These elastics 33 cause the barrier leg cuffs 32 to help form a seal around the legs and torso of a wearer. The leg elastics 34 extend at least partially between the front end edge 18 and the back end edge 20. The leg elastics 34 essentially cause portions of the absorbent article 10 proximate to the chassis side edges 22, 24 to help form a seal around the legs of the wearer. The leg elastics 34 may extend at least within the crotch region 14.

### Elastic Waistband

Referring to FIGS. 1 and 2, the absorbent article 10 may comprise one or more elastic waistbands 36. The elastic waistbands 36 may be positioned on the garment-facing surface 2 or the wearer-facing surface 4. As an example, a first elastic waistband 36 may be present in the front waist region 12 near the front belt end edge 18 and a second elastic waistband 36 may be present in the back waist region 16 near the back end edge 20. The elastic waistbands 36 may aid in sealing the absorbent article 10 around a waist of a wearer and at least inhibiting bodily exudates from escaping the absorbent article 10 through the waist opening circumference. In some instances, an elastic waistband may fully surround the waist opening circumference of an absorbent article. The waistband may comprise elastic strands, elastic films, or combinations thereof. The waistband may be a stretch laminate within the scope of the present disclosure and may be ultrasonically bonded.

### Acquisition Materials

Referring to FIGS. 1, 2, 7, and 8, an acquisition layer comprising one or more acquisition materials 38 may be present at least partially intermediate the topsheet 26 and the absorbent core 30. The acquisition materials 38 are typically hydrophilic materials that provide significant wicking of bodily exudates. These materials may dewater the topsheet 26 and quickly move bodily exudates into the absorbent core 30. The acquisition materials 38 may comprise one or more nonwoven materials, foams, formed films, apertured formed films, cellulosic materials, cross-linked cellulosic materials, air laid cellulosic nonwoven materials, spunlace materials, or combinations thereof, for example. In some instances, portions of the acquisition materials 38 may extend through portions of the topsheet 26, portions of the topsheet 26 may extend through portions of the acquisition materials 38, and/or the topsheet 26 may be nested with the acquisition materials 38. Typically, an acquisition material 38 may have a width and length that are smaller than the width and length of the topsheet 26. The acquisition material may be a secondary topsheet in the feminine pad context. The acquisition material may have one or more channels as described above with reference to the absorbent core 30 (including the embossed version). The channels in the acquisition material may align or not align with channels in the absorbent core 30. In an example, a first acquisition material may comprise a nonwoven material and as second acquisition material may comprise a cross-linked cellulosic material.

### Landing Zone

Referring to FIGS. 1 and 2, the absorbent article 10 may have a landing zone area 44 that is formed in a portion of the garment-facing surface 2 of the outer cover material 40. The landing zone area 44 may be in the back waist region 16 if the absorbent article 10 fastens from front to back or may be in the front waist region 12 if the absorbent article 10 fastens back to front. In some instances, the landing zone 44 may be or may comprise one or more discrete nonwoven materials that are attached to a portion of the outer cover material 40 in the front waist region 12 or the back waist region 16 depending upon whether the absorbent article fastens in the front or the back. In essence, the landing zone 44 is configured to receive the fasteners 46 and may comprise, for example, a plurality of loops configured to be engaged with, a plurality of hooks on the fasteners 46, or vice versa. The landing zone may comprise a nonwoven with enough fiber loops to enable adequate fastening.

### Wetness Indicator/Graphics

Referring to FIG. 1, the absorbent articles 10 of the present disclosure may comprise graphics 78 and/or wetness indicators 80 that are visible from the garment-facing surface 2. The graphics 78 may be printed on the landing zone 40, the backsheet 28, and/or at other locations. The wetness indicators 80 are typically applied to the absorbent core facing side of the backsheet 28, so that they can be contacted by bodily exudates within the absorbent core 30. In some instances, the wetness indicators 80 may form portions of the graphics 78. For example, a wetness indicator may appear or disappear and create/remove a character within some graphics. In other instances, the wetness indicators 80 may coordinate (e.g., same design, same pattern, same color) or not coordinate with the graphics 78. The wetness indicators may be slot coated, gravure printed or digitally printed onto a carrier substrate. The indication action, usually a color change, may be pH-sensitive (blue to green to yellow, blue to yellow, other) or may be thermochromic (temp-sensitive).

### Front and Back Ears

Referring to FIGS. 1 and 2, as referenced above, the absorbent article 10 may have front and/or back ears 47, 42 in a taped diaper context. Only one set of ears may be required in most taped diapers. The single set of ears may comprise fasteners 46 configured to engage the landing zone or landing zone area 44. If two sets of ears are provided, in most instances, only one set of the ears may have fasteners 46, with the other set being free of fasteners. The ears, or portions thereof, may be elastic or may have elastic panels. In an example, an elastic film or elastic strands may be positioned intermediate a first nonwoven material and a second nonwoven material. The elastic film may or may not be apertured. The ears may be shaped. The ears may be integral (e.g., extension of the outer cover material 40, the backsheet 28, and/or the topsheet 26) or may be discrete components attached to a chassis 52 of the absorbent article on a wearer-facing surface 4, on the garment-facing surface 2, or intermediate the two surfaces 4, 2. Additionally or alternatively, any of the ears 42, 47 may comprise a stretch laminate as described hereinafter. The front and/or back ear comprises a Length Ratio of about 3 or less, or about 2.95 or less, or from about 1 to about 3, or from about 1.75 to about 3, or from about 1 to about 2.5 as determined by the Tensile Test Method herein, reciting for each range every 0.05 interval therein. Forming an ear with such Length Ratios decreases the potential for roping within the ear. Further, the specified Length Ratios result in increased strength in the ear.

### Masking Layer

One or more masking layers or materials may be provided in the absorbent articles 10. A masking layer may be a layer that provides a cushiony feel when the absorbent article is touched from the garment-facing surface 2 or the wearer-facing surface 4. The masking layer may “mask” a grainy feel potentially caused by the absorbent material 72, such as superabsorbent polymers. The masking layer may “mask” bodily exudates from being visible when viewing the wearer-facing surface 4 or the garment-facing surface 2 of the absorbent article 10. The masking layer may have a basis weight in the range of about 15 gsm to about 50 gsm or about 15 gsm to about 40 gsm. The masking layer may comprise one or more nonwoven materials (e.g., a hydroentangled nonwoven material), foams, pulp layers, and/or other suitable materials. The masking layer may be the outer cover material 40. The masking layer may be the layer forming the garment-facing side or the wearer-facing side of the core bag 74. The masking layer may be a separate material positioned intermediate the garment-facing side of the core bag 74 and the liquid impermeable backsheet 28.

### Sensors

Referring again to FIG. 1, the absorbent articles of the present disclosure may comprise a sensor system 82 for monitoring changes within the absorbent article 10. The sensor system 82 may be discrete from or integral with the absorbent article 10. The absorbent article 10 may comprise sensors that can sense various aspects of the absorbent article 10 associated with insults of bodily exudates such as urine and/or BM (e.g., the sensor system 82 may sense variations in temperature, humidity, presence of ammonia or urea, various vapor components of the exudates (urine and feces), changes in moisture vapor transmission through the absorbent articles garment-facing layer, changes in translucence of the garment-facing layer, and/or color changes through the garment-facing layer). Additionally, the sensor system 82 may sense components of urine, such as ammonia or urea and/or byproducts resulting from reactions of these components with the absorbent article 10. The sensor system 82 may sense byproducts that are produced when urine mixes with other components of the absorbent article 10 (e.g., adhesives, agm). The components or byproducts being sensed may be present as vapors that may pass through the garment-facing layer. It may also be desirable to place reactants in the absorbent article that change state (e.g. color, temperature) or create a measurable byproduct when mixed with urine or BM. The sensor system 82 may also sense changes in pH, pressure, odor, the presence of gas, blood, a chemical marker or a biological marker or combinations thereof. The sensor system 82 may have a component on or proximate to the absorbent article that transmits a signal to a receiver more distal from the absorbent article, such as an iPhone, for example. The receiver may output a result to communicate to the caregiver a condition of the absorbent article 10. In other instances, a receiver may not be provided, but instead the condition of the absorbent article 10 may be visually or audibly apparent from the sensor on the absorbent article.

### Bio-Based Content for Components

Components of the absorbent articles described herein may at least partially be comprised of bio-based content as described in U.S. Pat. Appl. No. 2007/0219521A1. For example, the superabsorbent polymer component may be bio-based via their derivation from bio-based acrylic acid. Bio-based acrylic acid and methods of production are further described in U.S. Pat. Appl. Pub. No. 2007/0219521 and U.S. Pat. Nos. 8,703,450; 9,630,901 and 9,822,197. Other components, for example nonwoven and film components, may comprise bio-based polyolefin materials. Bio-based polyolefins are further discussed in U.S. Pat. Appl. Pub. Nos. 2011/0139657, 2011/0139658, 2011/0152812, and 2016/0206774, and U.S. Pat. No. 9,169,366. Example bio-based polyolefins for use in the present disclosure comprise polymers available under the designations SHA7260™, SHE150™, or SGM9450F™ (all available from Braskem S.A.).

An absorbent article component may comprise a bio-based content value from about 10% to about 100%, from about 25% to about 100%, from about 40% to about 100%, from about 50% to about 100%, from about 75% to about 100%, or from about 90% to about 100%, for example, using ASTM D6866-10, method B.

### Recycle Friendly and Bio-Based Absorbent Articles

Components of the absorbent articles described herein may be recycled for other uses, whether they are formed, at least in part, from recyclable materials. Examples of absorbent article materials that may be recycled are nonwovens, films, fluff pulp, and superabsorbent polymers. The recycling process may use an autoclave for sterilizing the absorbent articles, after which the absorbent articles may be shredded and separated into different byproduct streams. Example byproduct streams may comprise plastic, superabsorbent polymer, and cellulose fiber, such as pulp. These byproduct streams may be used in the production of fertilizers, plastic articles of manufacture, paper products, viscose, construction materials, absorbent pads for pets or on hospital beds, and/or for other uses. Further details regarding absorbent articles that aid in recycling, designs of recycle friendly diapers, and designs of recycle friendly and bio-based component diapers, are disclosed in U.S. Pat. Appl. Publ. No. 2019/0192723, published on Jun. 27, 2019.

### Stretch Laminate

Various elements, particularly elastic side members, of the absorbent articles 10 described herein may comprise a stretch laminate. For example, any of the belts 54 and 56 and/or any of the ears 42, 47 may comprise a stretch laminate as described hereinafter. The waistbands may also comprise a stretch laminate. Such laminates may include an elastomeric layer that provides extensibility to the laminate and one or more outer layers that is less stretchable but suitable for providing durability and desirable tactile properties. In this way, the laminate permits a component of an absorbent article to closely and comfortably contact the wearer while providing desirable exterior qualities.

FIGS. 12A-12F are a cross-sectional views of various stretch laminates 90. As shown in FIG. 12A, a stretch laminate 90 may comprise a first cover layer 100 and an elastomeric film layer 300 joined via one or more ultrasonic bonds 400. The elastomeric film layer 300 may have one or more skins, such as a first skin 301 providing first surface and a second skin 302 providing second surface. As shown in FIG. 12B, a stretch laminate 90 may comprise a first cover layer 100 and a second cover layer 200 with an elastomeric film layer 300 sandwiched therebetween in a facing relationship to both the first cover layer 100 and the second cover layer 200. All three layers may be joined via one or more ultrasonic bonds 400. As shown in FIGS. 12C and 12D, all or a portion of the first cover layer 100 may comprise one or more layers, such as a first layer 101 and second layer 102, which may have the same composition or different compositions. Similarly, all or a portion of the second cover layer 200 may comprise one or more layers, such as a first layer 201 and second layer 202, which may have the same composition or different compositions. As shown in FIGS. 12E and 12F, a portion of the first cover layer 100 or the second cover layer 200 may be folded over to provide a multi-layered structure on all or a portion of the opposite side of stretch laminate 90.

Elastomeric film layer 300 of stretch laminate 90 may include a single layer or multiple layers of one or more materials that are elastically extensible. The elastically extensible material(s) may be between about 10 μm and about 100 μm, or between about 20 μm and about 60 μm, or between about 30 μm and about 50 μm, or in some embodiments, about 40 μm, in thickness. The 20 elastically extensible material(s) may comprise an elastomeric polyolefin, and in some embodiments, a polyolefin (POE) blown film.

The elastically extensible material may comprise modifying resins.

The elastically extensible material may comprise a variety of additives. Suitable additives including, but not limited to, stabilizers, antioxidants, and bacteriostats may be employed to prevent thermal, oxidative, and bio-chemical degradation of the elastically extensible material. Additives may account for about 0.01% to about 60% of the total weight of the elastically extensible material. In other embodiments, the composition comprises from about 0.01% to about 25%. In other suitable embodiments, the elastically extensible material comprises from about 0.01% to about 10% by weight, of additives.

The elastically extensible material may comprise various stabilizers and antioxidants that are well known in the art and include high molecular weight hindered phenols (i.e., phenolic compounds with sterically bulky radicals in proximity to the hydroxyl group), multifunctional phenols (i.e., phenolic compounds with sulfur and phosphorous containing groups), phosphates such as tris-(p-nonylphenyl)-phosphite, hindered amines, and combinations thereof. Proprietary commercial stabilizers and/or antioxidants are available under a number of trade names including a variety of Wingstay®, Tinuvin® and Irganox® products.

The elastically extensible material may comprise various bacteriostats that are known in the art. Examples of suitable bacteriostats include benzoates, phenols, aldehydes, halogen containing compounds, nitrogen compounds, and metal-containing compounds such as mercurials, zinc compounds and tin compounds. A representative example is available under the trade designation Irgasan Pa. from Ciba Specialty Chemical Corporation of Tarrytown, N.Y.

The elastically extensible material may comprise viscosity modifiers, processing aids, slip agents or anti-block agents. Processing aids include processing oils, which are well known in the art and include synthetic and natural oils, naphthenic oils, paraffinic oils, olefin oligomers and low molecular weight polymers, vegetable oils, animal oils, and derivatives of such including hydrogenated versions. Processing oils also may incorporate combinations of such oils. Mineral oil may be used as a processing oil. Viscosity modifiers are also well known in the art. For example, petroleum derived waxes can be used to reduce the viscosity of the slow recovery elastomer in thermal processing. Suitable waxes include low number-average molecular weight (e.g., 0.6-6.0 kilo Daltons) polyethylene; petroleum waxes such as paraffin wax and microcrystalline wax; atactic polypropylene; synthetic waxes made by polymerizing carbon monoxide and hydrogen such as Fischer-Tropsch wax; and polyolefin waxes.

The desirability and noticeability of many of stretch laminate features have been tested with consumers. Nonwoven choice can influence the appearance of a bond pattern as well as change the tactile softness of the laminate. Changes in ultrasonic and nonwoven bond pattern can change the texture of the stretch laminate, both tactically and visually. In short, varying these parameters in a laminate can make clear differentiation that is consumer noticeable and appreciated. Some further variations are detailed hereinafter.

### Skin

Exemplary elastomeric film layers 300 that are useful in the stretch laminates 90 detailed herein (i.e., an elastically extensible material with at least one skin disposed on the surface of the elastically extensible material) include M18-1117 and M18-1361 elastomeric films commercially available from Clopay Corporation of Cincinnati, Ohio; K11-815 and CEX-826 elastomeric films commercially available from Tredegar Film Products of Richmond, Virginia; and elastomeric films commercially available from Mondi Gronau GmbH of Gronau, Germany. These exemplary elastomeric films may include a single layer of elastically extensible material with a skin 301, 302 disposed on both surfaces of the material. Other elastomeric film layers applicable to the stretch laminates detailed herein need not have a skin 301, 302 on both surfaces of the material and may instead have no skin or a skin on only one surface.

### Nonwoven Material

The first cover layer 100 and the cover layer material 200, as well as any layers 101, 102, 201, 202 that make up either material, may include any suitable nonwoven material or combination of nonwoven materials, including but not limited to, spun only or spun meltblown combinations, such as SM (spunbond meltblown), SMS (spunbond meltblown spunbond), SMMS (spunbond meltblown meltblown spunbond) nonwovens, SSMMS (spunbond spunbond meltblown meltblown spunbound), hydroentangled nonwovens and softbond nonwovens. The nonwoven materials may also include carded nonwovens, such as those specially designed and manufactured to be compatible with an activation (e.g., ring-rolling) process. One exemplary nonwoven material is a carded nonwoven made from a polypropylene homopolymer. The spunbounds may also be specially designed and/or manufactured to be compatible with an activation process. However, it is believed that through the use of the elastomeric film according to the present disclosure, greater flexibility in the design choices may be achieved. For example, spunbounds may be selected for applications where only carded nonwovens were used in the past, or thinner elastomeric films may be used with the carded nonwovens. Other improvements in design flexibility will also be recognized by the skilled practitioner. For example, in some embodiments, the cover layer(s) may be extensible nonwovens and may or may not need to undergo an activation process in order to impart extensibility to the stretch laminate.

The basis weight of the nonwoven material may be less than about 30 gsm. In fact, according to certain embodiments, the basis weight may be less than about 27 gsm. In other embodiments, the basis weight may be less than about 25 gsm. In still other embodiments, the nonwoven material may have a basis weight of less than about 24 gsm. The nonwoven materials may also include additives, such as, for example, CaCO₃. Woven or knitted fabrics may also be used as cover layers 100, 200 in embodiments of the stretch laminates 90 detailed herein.

The ultrasonic bonds 400 preferably eliminate the need for any adhesives, but adhesives may be employed to join the layers 100, 200, 300 of the stretch laminate 90. Adhesives may be selected from any adhesives known to provide suitable attachment between elastomeric film layer 300 and cover layers 100, 200. In some embodiments, the adhesive may be a hot melt adhesive with a basis weight of less than about 15 gsm. According to one embodiment, the adhesive may be H2031 adhesive commercially available from Bostik Inc. of Middleton, Massachusetts. One characteristic of this adhesive is that, at 23° C., this adhesive has significant pressure-sensitive character useful for making a stretch laminate by hand. However, this adhesive is also suitable for use in fabricating stretch laminates from the elastomeric films and cover layers listed above using conventional stretch laminate manufacturing equipment, such equipment being well known in the art.

sPre-Activation

Elastomeric film layer 300 may be mechanically pre-activated before attachment to at least one cover layer 100, 200. For example, elastomeric film layer 300 may be pre-activated by being stretched transversely to its web direction by more than 50% (i.e., strain>50%). In some embodiments, an expansion by about 100% to about 500% occurs in relation to the starting width of elastomeric film layer 300. In alternate embodiments, elastomeric film layer 300 may be stretched in the web direction, stretched a direction other than the web direction or transverse to the web direction, or a combination of directions. The term “stretching” is to point to the fact that the expansion of elastomeric film layer 300 is not completely reversible and that a non-elastic fraction results in the film having a larger width following pre-activation (i.e., the elastomeric film does not have 100% recovery, and therefore has a percent set value). After expansion, elastomeric film layer 300 retracts and has a width that may be larger by about 10% to about 30% in relation to a starting width of the film. In other words, after the pre-activation expansion and retraction detailed below, elastomeric film layer 300 may exhibit a set of about 10% to about 30%.

According to various embodiments in which elastomeric film layer 300 includes both an elastically extensible material and at least one skin disposed on the elastically extensible material, the pre-activation process may physically alter these materials differently, for example, because these materials have different elasticity and recovery properties. During pre-activation, the skin 301 and/or 302 and the elastically extensible material are similarly stretched (i.e., put under similar strain). However, after stretching, the skin and the elastically extensible material will retract and recover differently (i.e., have different set values). In comparison with the elastically extensible material, the skin is less elastic and therefore will have less recovery after stretching, a.k.a., a higher set value. The skin is also much thinner than the elastically extensible material, so when the thicker elastically extensible material retracts and recovers after pre-activation stretching, it will force the attached skin to retract with it. But because the skin cannot recover as much as the elastically extensible material, the skin buckles and wrinkles. Accordingly, the cross-sectional profile and the top view appearance of elastomeric film layer 300 are modified after a pre-activation process.

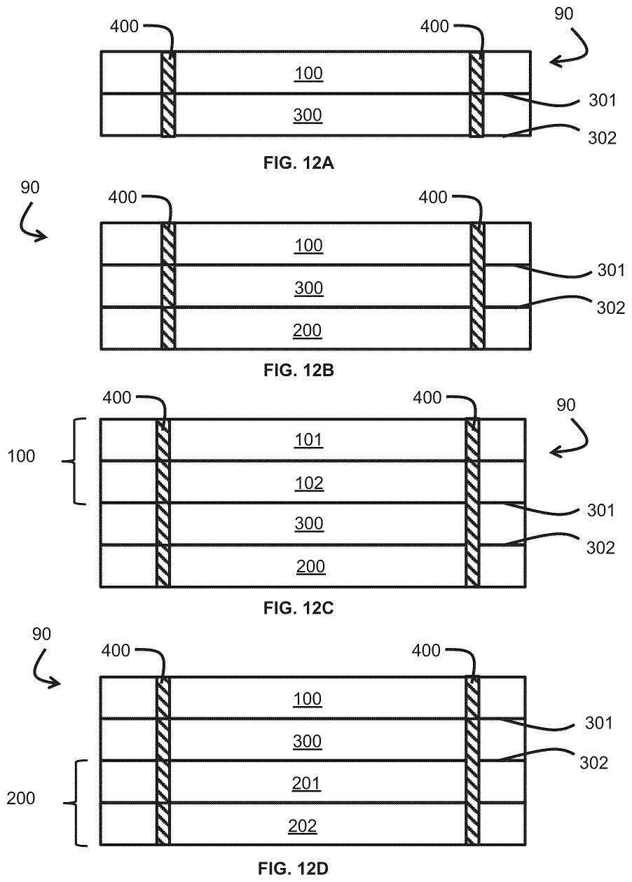

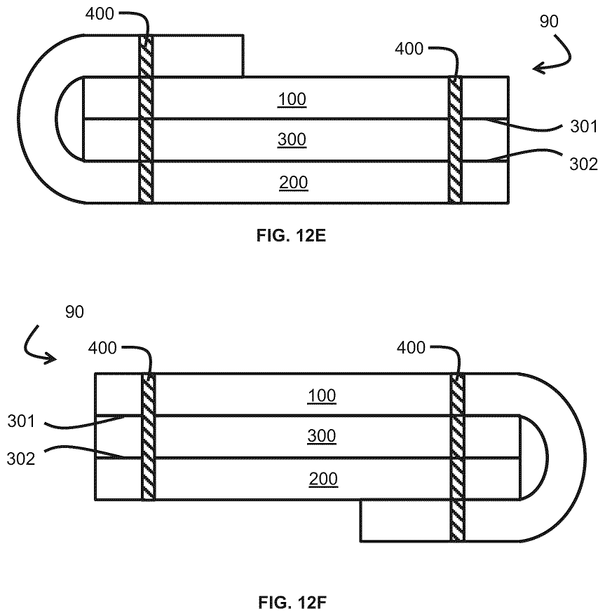

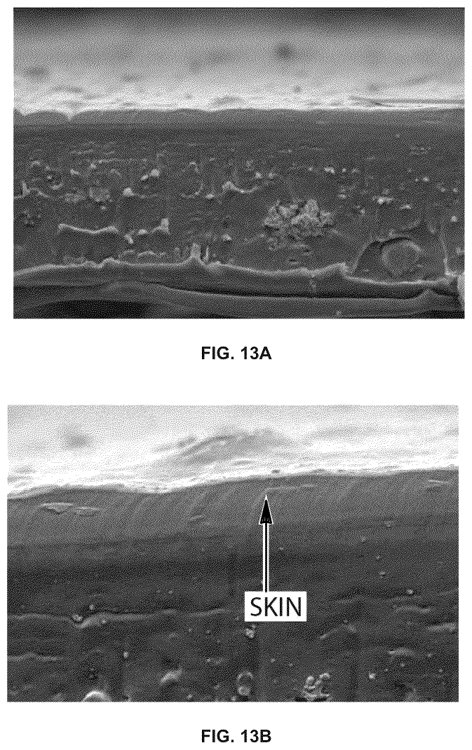

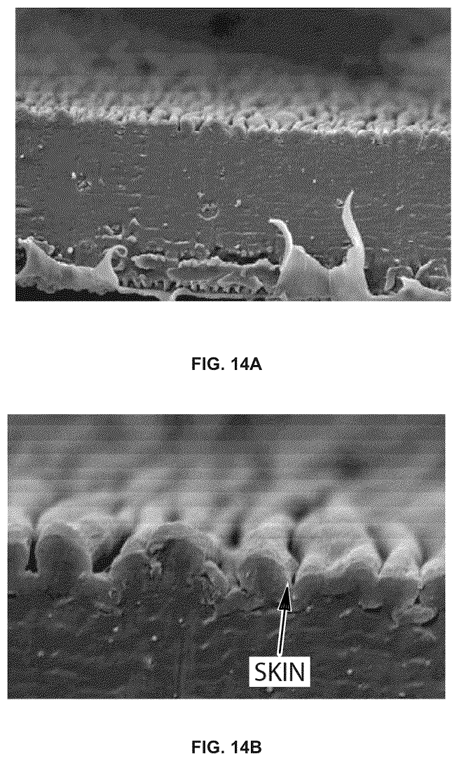

FIGS. 13A, 13B, 14A, and 14B are SEM photomicrographs of magnified cross-sections of elastomeric films. These SEM photomicrographs, as well as the other SEM photomicrographs included herein, were taken with a scanning electron microscope (Hitachi Model 3500). The information to calculate specific magnifications and distances is included in each individual SEM photomicrograph along the bottom of the frame. FIG. 13A is a SEM photomicrograph taken at approximately 900× magnification showing a cross-sectional view of a portion of an elastomeric film that has not been pre-activated. The skins are the thin strips of contrasting material at the top and the bottom of the cross-section, with the thicker elastically extensible material between the skins. The skin at the top of the cross-section is easier to discern due to the cross-section being cut cleaner in that region. Without pre-activation, the skins, and thus the outer surfaces of the elastomeric film, are substantially smooth in a cross-sectional view. FIG. 13B is a higher magnification image (approx. 3500× magnification) of the skin at the top of cross-section shown in the SEM photomicrograph of FIG. 13A.

FIG. 14A is a SEM photomicrograph taken at approximately 900X magnification showing a cross-sectional view of a portion of an elastomeric film that has been pre-activated. Again, the skins are the thin strips of contrasting material at the top and the bottom of the cross-section, with the thicker elastically extensible material between the skins. With pre-activation, the skins, and thus the outer surfaces of the elastomeric film, are wrinkled in a cross sectional view. FIG. 14B is a higher magnification image (approx. 3500× magnification) of the skin at the top off the cross-section shown in the SEM photomicrograph of FIG. 14A.

FIGS. 14A and 14B show that after pre-activation, the skin 301 of elastomeric film layer 300 includes a plurality of wrinkles having hills and furrows. For example, as shown in the non-limiting sample photographed in FIG. 14B, there are approximately six hills and six furrows of varying size within the pictured approximately 35 μm of length taken along the cross-sectional profile of the pre-activated elastomeric film. This is in comparison to FIG. 13B, in which there are no hills and no furrows within the pictured approximately 35 μm of length taken along the cross-sectional profile of an elastomeric film that was not pre-activated. However, as visible on the top surface of the elastomeric film shown in FIG. 14B, one or more random hills and/or furrows may be present within a particular length of cross-sectional profile of an elastomeric film that was not pre-activated. These random hills and/or furrows are due to irregularities in the surface of the elastomeric film. Such random hills and/or furrows should not be confused with the hills and furrows of the plurality of wrinkles that are intentionally formed in an elastomeric film through a mechanical pre-activation process.

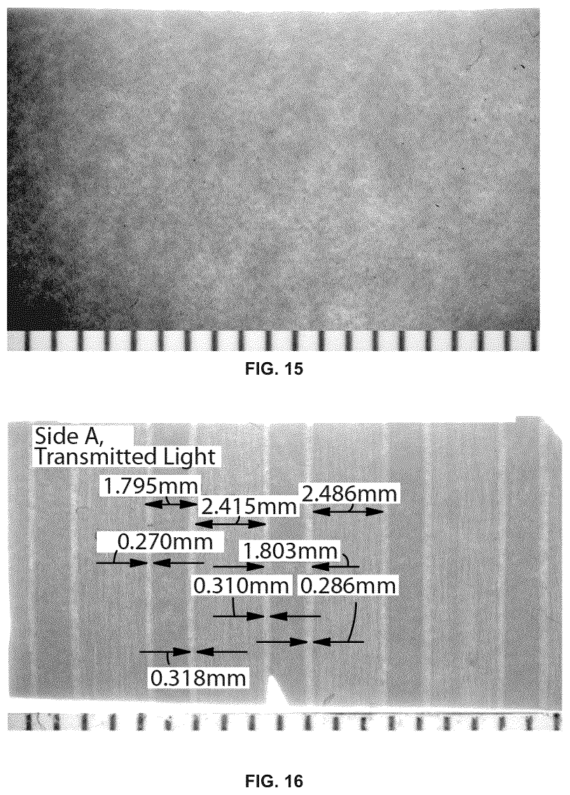

FIGS. 15 and 16 are transmitted light photomicrographs of magnified top views of elastomeric films. The transmitted light photomicrographs were taken in color using a Nikon SMZ 1500 Stereo Light Microscope equipped with an Evolution Mp5C Digital camera with white light shining underneath the elastomeric film samples. The blue scale marks at the bottoms of FIGS. 15 and 16 are in millimeters. This scale can be used to calculate specific magnifications and distances in the transmitted light photomicrographs. FIG. 15 is a transmitted light photomicrograph showing a top view of a portion of an elastomeric film that has not been pre-activated. Without pre-activation, the viewable outer surface of the elastomeric film (i.e., the top view of the skin), has no discernible stripes and is uniform in appearance. FIG. 16 is a transmitted light photomicrograph showing a top view of a portion of an elastomeric film that has been pre-activated. With pre-activation, the top view of the skin includes a plurality of stripes in varying thicknesses that relate to the size and pitch of the intermeshing discs of the mechanical pre-activation means. The stripes, referred to herein as activation stripes, indicate zones in the pre-activated elastomeric film in which there was a particular range of stretching during the pre-activation process. For example, as shown in non-limiting sample photographed in FIG. 16, there are medium thickness darker blue stripes indicative of a heavier intensity skin wrinkling, large thickness light blue stripes indicative of medium intensity skin wrinkling, and thin white stripes indicative of lower intensity skin wrinkling.

In addition, after preactivation, but before utilizing elastomeric film layer 300 in the fabrication of stretch laminate 90, the film layer 300 may optionally be printed with an image or motif that may show through the cover layers 100, 200 of the stretch laminate 90. The ink or other pigment utilized in printing will be deposited on the hills and into the furrows of the wrinkles of the pre-activated elastomeric film. Ink deposited onto the textured surface of a pre-activated elastomeric film allows for more contact surface area between the elastomeric film and the ink. Accordingly, when printing on a pre-activated elastomeric film, there is an image that is more strongly set on the film when compared to an image printed on the much smoother surface of an elastomeric film that has not been pre-activated.

Moreover, when stretch laminate 90 includes a pre-activated (and subsequently printed) elastomeric film that is mechanically, a non-distorted printed image on the film is evenly and reversibly stretched along with it. This is because before the image was printed on the pre-activated elastomeric film, a significant portion, or the entire, non-elastic fraction of elastomeric film 90 has already been removed in the pre-activation process. In other words, the set had been removed from elastomeric film layer 300 before printing. Therefore, the printed image will not substantially distort further with the later activation of stretch laminate 90, or in additional stretching of the laminate by a user. In contrast, if an image or motif were printed on an elastomeric film that was not pre-activated, and that printed film was then used in fabricating a stretch laminate, and then the stretch laminate was mechanically activated, the desired image would be distorted in the final activated stretch laminate. This is because the set of the elastomeric film was not removed prior to the printing process, and such set would be removed from the elastomeric film in the mechanical activation of the fabricated stretch laminate, thus distorting the original printed image. Likewise, if an elastomeric film is printed and then subsequently preactivated, the set of the elastomeric film will not be removed prior to the printing process, and such set would be removed from the elastomeric film in the pre-activation process, thus distorting the original printed image.

And in another embodiment, a pre-activated elastomeric film may be stretched again during the printing of the film. The printed film is then relaxed and used in fabrication and activation of the stretch laminate. The resulting activated stretch laminate has an image or motif that is aesthetically pleasant when the stretch laminate is in a stretched condition during use (e.g., when a user stretches the stretch laminate in application or removal of an absorbent article).

In embodiments of stretch laminates that include a elastomeric film that is pre-activated and subsequently printed, the ink or other pigment utilized in printing will be deposited on the hills and into the furrows of the wrinkles of the film. As detailed above, ink deposited onto the textured surface of a pre-activated elastomeric film will more strongly set on the film due to the additional contact surface area between the elastomeric film and the ink (in comparison to ink disposed on a elastomeric film that has not been pre-activated).

In addition, pre-activating an elastomeric film also lowers the force needed to later stretch the film (versus a non-activated film). This helps the later mechanical activation of the stretch laminate because the load required to activate a stretch laminate that is made with pre-activated film will be lower (versus a non-activated film).

### Stretch Laminate Fabrication Method

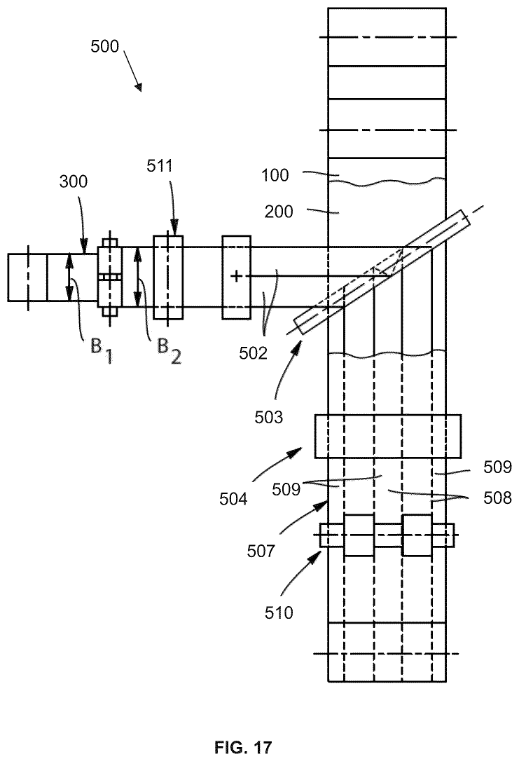

The schematic illustration of FIG. 17 details an exemplary embodiment of a method 500 for fabricating the stretch laminates 90 detailed herein. The method 500 includes providing and pre-activating an elastomeric film 300. Elastomeric film 300 is mechanically pre-activated by stretching the film transverse to its web direction by more than 50%. In some embodiments, an expansion by about 100% to about 500% occurs in relation to the starting width of elastomeric film 300. The term “stretching” is to point to the fact that the expansion of elastomeric film 300 is not completely reversible and that a non-elastic fraction results in the film having a larger width following retraction (i.e., reverse expansion). After expansion, elastomeric film 300 retracts and has a width B₂ that is larger by about 10% to about 30% in relation to a starting width B₁ of the film. Accordingly, elastomeric film 300 has a set of about 10% to about 30% resulting from the pre-activation process.

For the pre-activation process, elastomeric film 300 may be guided through a system of intermeshing profile rollers, each roller including disk packets having a plurality of intermeshing disks that are situated on an axis (i.e., a ring rolling process). Elastomeric film 300 is transversely stretched by the intermeshing disk packets. The stretching may be uniform or varied over the width of the film. The pre-activation process can be carried out at varying pitch and or varying depths of engagement. The pre-activation process can also be carried out in machine direction, or in any other direction. The pre-activation of elastomeric film 1 has a positive effect on the stretching force profile and helps allow for an easy stretching action of the fabricated stretch laminate over a large expansion area. Further, the recovery of the stretch laminate can also be improved by pre-activating elastomeric film 300. The recovery is the ability of a stretch laminate to return to original size after it has been stretched to its expansion limit. The increased recovery of elastomeric film 300 after the pre-activation process is due to the removal of an amount of set from the film.

After preactivation, but before cutting elastomeric film 300 into film strips 502, the film may optionally be printed in a printing station 511 with an image or motif that may show through the cover layers of the stretch laminate. Any known continuous printing methods can be used for printing the elastomeric film 300. Non-limiting exemplary printing methods include digital printing, inkjet printing, and rotary printing methods, in particular, flexography. As a non-limiting example, the printed image or motif can be a striped motif made of parallel colored stripes that extend in the web's longitudinal direction of elastomeric film 300.

The pre-activated, and optionally printed, film may optionally then be cut into film strips 502. The film strips 502 are guided across redirecting means 503 and supplied to laminating means 504 as parallel strips. Film strips 502 are then laminated in laminating means 504 between cover layers 100, 200, which are supplied above and below the film strips. Film strips 502 and cover layers 100, 200 may be glued together or connected to each other by thermal means, such as ultrasonic bonding, to form composite material 507 (i.e., an embodiment of the stretch laminate materials detailed herein). As illustrated in FIG. 17, film strips 502 are laminated at a distance relative to each other between cover layers 100, 200. Cover layers 100, 200 may, therefore, be directly connected to each other in the regions between film strips 502. Accordingly, elastic regions 508, as well as non-elastic regions 509, may optionally be created in composite material 507. The distance between film strips 502 can be adjusted by positioning the redirecting means. It is also contemplated that reinforcement strips may be laminated between film strips 502 to reinforce non-elastic regions 509 between the film strips.

Composite material 507 is then supplied to an activation means 510 in which the composite material is stretched at portions of elastic regions 508 transversely in relation to the direction of the web. For the stretching, composite material 507 may be guided through a nip between two profile rollers, each roller including at least two disk packets having a plurality of intermeshing disks that are situated on an axis. Composite material 507 is transversely stretched in places by the intermeshing disk packets. The regions in which composite 507 is stretched by the intermeshing disk packets are referred to as stretch zones. In the roller sections between and/or outside the disk packets, the profile rollers form a gap, through which composite 507 is guided though essentially without transverse stretching. The regions in which composite 507 is not stretched by the intermeshing disk packets are referred to as anchoring zones. In the stretch zones, the fibers of cover layers 100, 200 are modified and irreversibly stretched due to fiber tears and rearrangements. Accordingly, the expansion property of composite material 507 is improved in the stretch zones in the cross direction (i.e., transverse in relation to the longitudinal web direction). Following activation, when applying minimal force, composite material 507 is easily expandable in the cross direction to an expansion limit that is preset by the stretching of activation means 510.

When traditional nonwovens are utilized as the cover layers, any pre-activation of elastomeric film 300 cannot replace but can only supplement the mechanical activation of composite material 507. Accordingly, even when elastomeric film 300 is pre-activated, it is still necessary for composite material 507 to be stretched transversely relative to the direction of the web in the regions that are to be rendered elastic via laminated elastomeric film strips (i.e., stretch zones). However, there may be some embodiments of composite material 507 that use extensible nonwovens as the cover layers, and therefore it may not be necessary to activate the composite material.

In fabrication of the printed stretch laminate embodiments that are disclosed herein, elastomeric film 300 may optionally be printed with an image or motif, which shows through at least one of the cover layers 100, 200 of composite material 507. Due to the fact that elastomeric film 300 is provided with the imprint, correct alignment of the printed motif relative to the elastic region of composite material 507 is always ensured. In addition, when stretching composite material 507, the printed image is evenly and reversibly stretched along with it. Furthermore, the printed motif may show through on the front side as well as on the back side of composite material 507, such that the composite material is optically equally attractive on the front as well as the back side thereof.

### Ultrasonic Bond Shapes

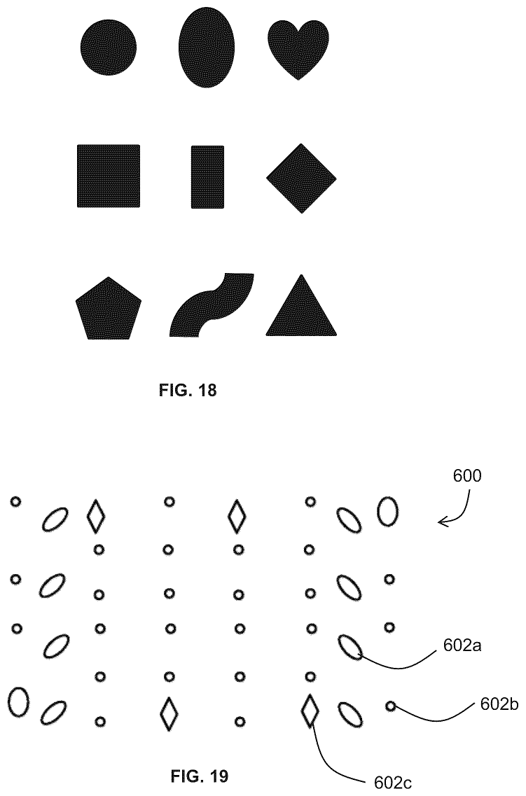

As shown in FIG. 18, individual ultrasonic bonds may be formed in a variety of shapes, including but not limited to curved shapes, straight-sided shapes, and combinations thereof. Examples of curved shapes include but are not limited to circles, ovals, and wavy lines. Examples of straight-sided shapes include but are not limited to triangles, squares, rectangles, diamonds, pentagons, hexagons, octagons, or any n-sided shapes. With respect to n-sided shapes, the shape may have any number of sides, for example, the shape could have from 3 to 12 sides. An example of a shape that is a combination of a curved shape and straight-side shape is a hearts. Providing various ultrasonic bond shapes may help to add visual appeal and to distinguish between various absorbent articles without compromising bond strength.

### Ultrasonic Bond Patterns

Groups of ultrasonic bonds may be arranged in units and the units may be arranged to form patterns. Patterns may be closed cell patterns, open cell patterns, or combinations thereof. A closed cell pattern comprises closed cell units, while an open cell pattern does not. A pattern may include a combination of regions having a closed cell pattern and other regions having an open cell pattern. FIG. 19 is a schematic illustration of an open cell bond pattern 600. The pattern 600 shown in FIG. 19 also comprises a plurality of ultrasonic bond shapes, including an oval bond shape 602a, a circular bond shape 602b, and a diamond bond shape 602c.

“Unit” is a smallest building block of a pattern, whose geometric arrangement defines pattern's characteristic imagery and whose repetition in space is necessary to re-construct the 30 pattern. A pattern may be formed from one or more units. A “repeating unit” is a unit that is substantially the same (i.e., slight variation of dimensions, shape, and/or size) or that is the same and is present multiple times within a pattern; said unit may be rotated, mirrored, or otherwise reoriented. The repeating unit is considered substantially the same if its size and/or shape is within 10% of another repeating unit.

“Closed cell unit” means a unit that is identifiable to the human eye with 20/20 vision from 12 inches away as a shape having a perimeter, the perimeter being formed by at least 5 bonds substantially surrounding an area free of permanent bonds that are less than about 3.5 mm Bond Separation Distance per the Bond Measurement Test Method. The perimeter may be formed by discontinuous bonding. For example, discrete bonds that are sufficiently small and/or close together that the viewer sees a shape substantially surrounded by a perimeter. Adjacent bonds along the perimeter of a closed cell unit are no more than about 3.5 mm Bond Separation Distance. Closed cell units may share bond sites with each other to form closed cell.

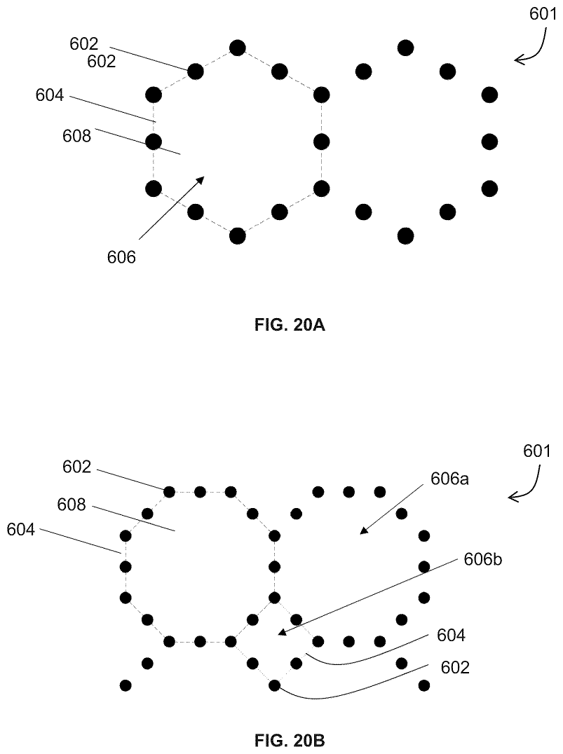

FIG. 20A is a schematic representation of an exemplary closed cell bond pattern 601 comprising a plurality of individual ultrasonic bonds 602. A perimeter 604 is shown as a dashed-line for illustrative purposes and to better understand the present disclosure; however, the dashed-line forms no part of the bond pattern. The perimeter 604 is formed by connecting adjacent, individual bonds 602 to surround a closed cell unit 606. The closed cell 606 unit includes an enclosed portion 608 that is substantially surrounded by the perimeter 604. It is to be appreciated that the perimeter 604 of a first closed cell unit 606 may form part of the perimeter of a second closed cell unit. It is also to be appreciated that a plurality of closed cell unit shapes may be employed in a single bond pattern. For example, FIG. 20A is a schematic representation of an exemplary closed cell bond pattern 601 comprising a plurality of closed cell unit shapes, including an octagonal closed cell unit 606a and a square closed cell unit 606b.

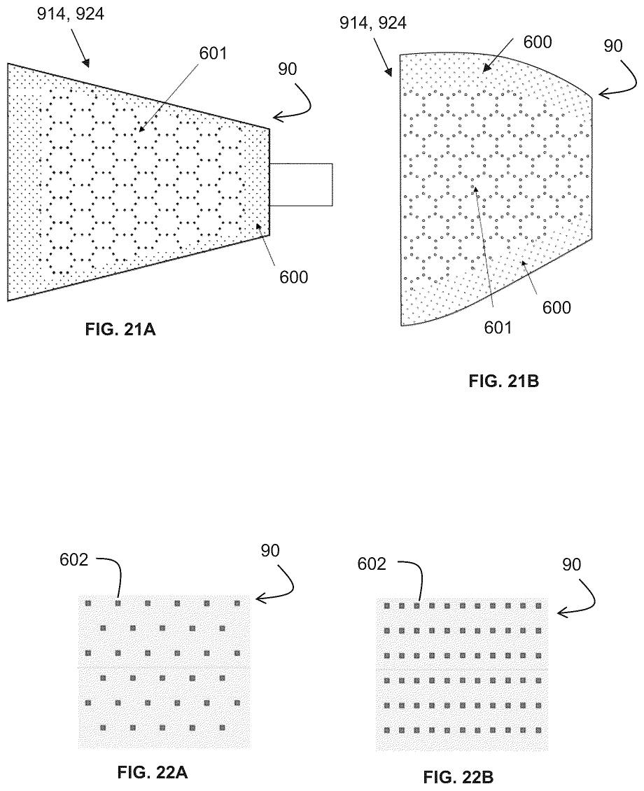

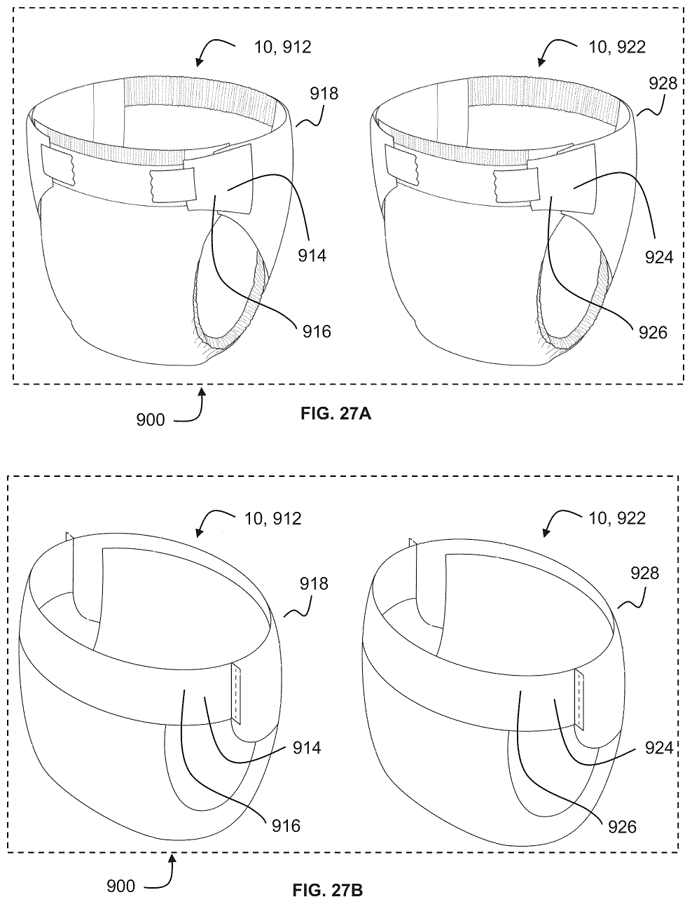

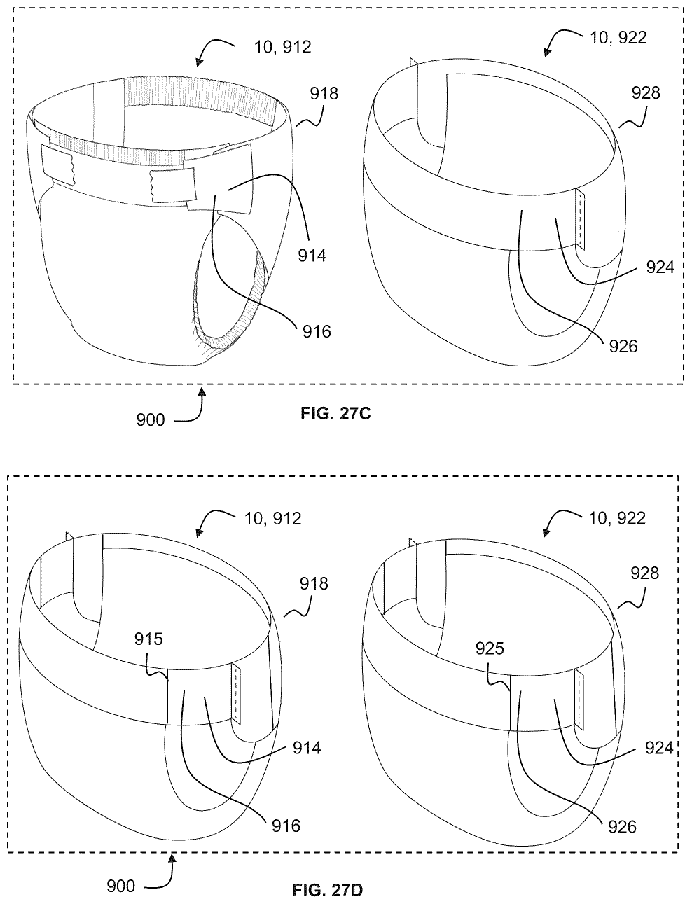

FIGS. 21A and 21B are plan views of an exemplary side members comprising stretch laminates 90 having more than one bond pattern. The exemplary side members may be a first elastic side member 914 or a second elastic side member, as illustrated in FIGS. 27A-27D. For example, laminates may comprise an open cell bond pattern 600 and a closed cell bond pattern 601. Additionally or alternatively, the second bond pattern may differ from the first bond pattern in at least one of shape of bonds, number of bonds, number of closed cell units (or absence of closed cell units), shape of repeat unit, enclosed area of closed cell units, bond density, percent bond area, and combinations thereof. The second bond pattern may be positioned outside of the first bond pattern, such that the two patterns may be in non-overlapping relationship. Such as illustrated in FIGS. 21A and 21B, the second bond pattern, shown as an open cell bond pattern 600, may be disposed along one or more edges of the laminate. In this way, the second bond pattern may at least partially surround or frame at least a portion of the first bond pattern, shown as a closed cell bond pattern 601 or the entire first bond pattern. The first bond pattern and the second bond pattern may also overlap in a transition zone. In embodiments with multiple bond patterns, such as a first bond pattern and a second bond pattern, the first bond pattern and the second bond pattern may also have different bond densities.

### Bond Density

“Bond density” refers to the number of bonds per unit area. FIGS. 22A and 22B are schematic illustrations of stretch laminates 90 having different bond densities, the stretch laminate 90 in FIG. 22A having a lower bond density than the laminate shown in FIG. 22B.

### Frangible Bonds

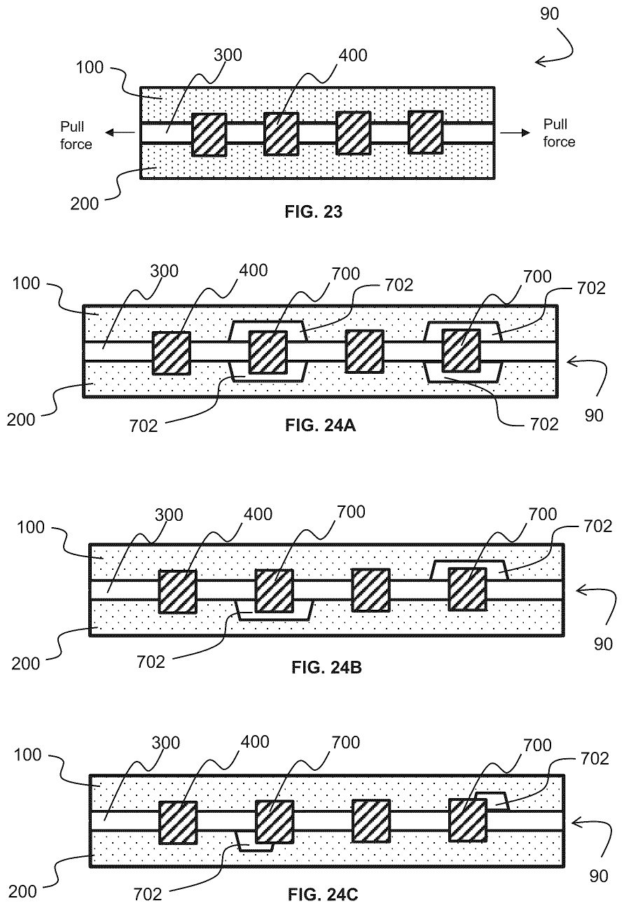

“Frangible bond” refers to an ultrasonic bond that is breakable upon being stretched. FIG. 23 is a schematic illustration of a stretch laminate 90 comprising an elastomeric film layer 300 sandwiched between a first cover layer 100 and a second cover layer 200. The layers are held together with a plurality of ultrasonic bonds 400. The stretch laminate 90 may be subjected to a lateral pull force. FIGS. 24A-24C is a schematic illustration of the stretch laminate 90 of FIG. 23 with various types of frangible bond sites 700 after being subjected to the lateral pull force. In FIG. 24A, the frangible bond sites 700 separate from both the first cover layer 100 and the second cover layer 200 at areas of detachment 702. In FIG. 24B, the frangible bond sites 700 separate from either the first cover layer 100 or the second cover layer 200 at areas of detachment 702. In FIG. 24C, the frangible bond sites 700 separate only partially from either the first cover layer 100 or the second cover layer 200 at areas of detachment 702.

### Cover Layer Primary Bond Pattern

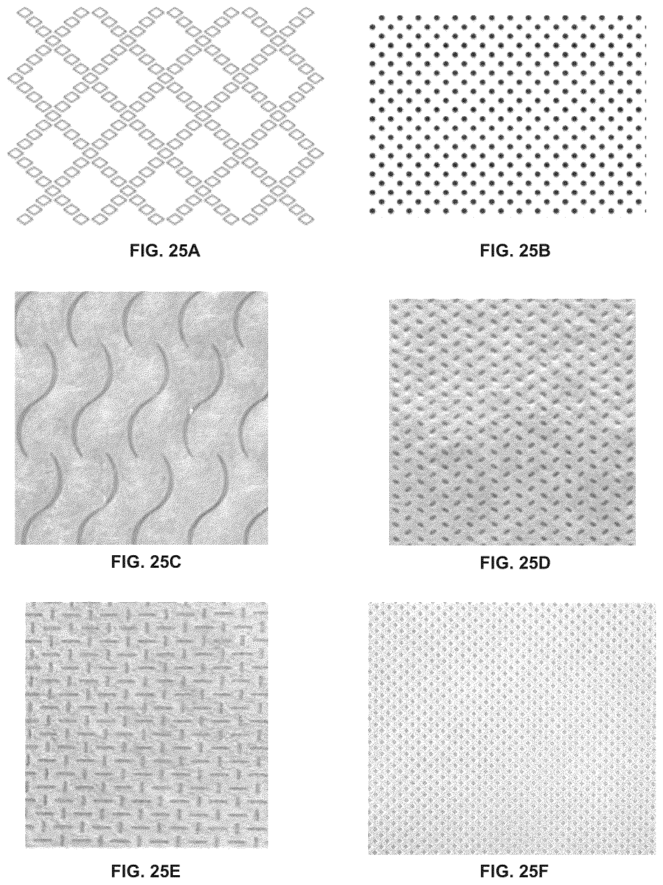

FIG. 25A-25F are schematic illustrations showing a variety of primary bond patterns for the cover layers that may be employed according to various embodiments. The bond patterns may be imprinted or embossed on a cover layer, such as first cover layer 100 or second cover layer 200 as described herein. As described herein, the cover layers may comprise a nonwoven material, in which case the primary bond pattern may be referred to as a nonwoven bond pattern. Such patterns may provide appealing and/or distinctive textures and appearances to stretch laminates.

Laminate with Slits and Cuts

In some configurations, it may be desirable to provide a stretch laminate or a side member comprising a stretch laminate with different zones of performance characteristics. Such enhanced properties may include breathability, softness, strength, thickness, uniformity in rugosities, modulus, aesthetic enhancements, tear resistance, combinations of any of the foregoing and/or zones having differing values of any of the foregoing features. According to various embodiments, the stretch laminates and/or side members comprising stretch laminates may comprise: embossing, apertures, perforations, slits, melted material or coatings, compressed material, secondary bonds that are disposed apart from the chassis attachment bond, plastic deformation, and folds.

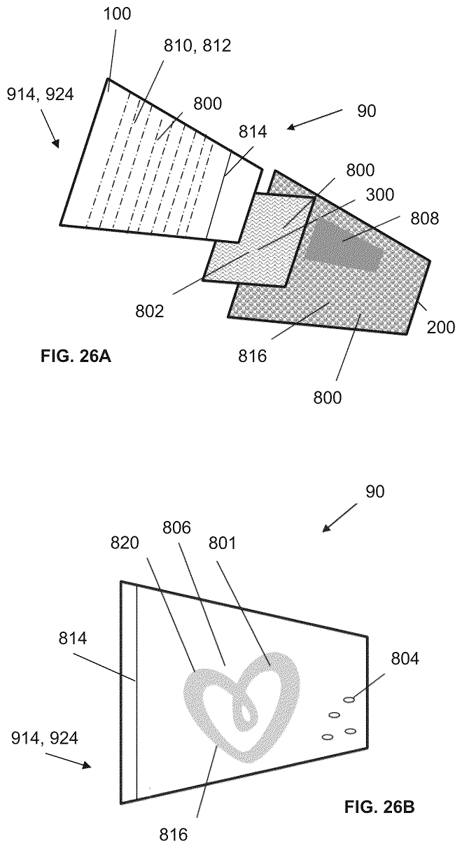

FIG. 26A is an exploded perspective view of an exemplary side member schematically illustrating exemplary surface modifications. The exemplary side members may be a first elastic side member 914 or a second elastic side member, as illustrated in FIGS. 27A-27D. The surface modification(s) may be made to a precursor material prior to lamination. One or more surface modifications 800 (also referred to as morphological features) are illustrated in one or more layers of the laminate. Surface modifications 800 may comprise embossing 802, cuts 804 (e.g., apertures, perforations, slits), melted material or coatings 806, compressed material 808, plastic deformation 810 (e.g., activation stripes 812), folds 814, post-formation bonds 816 (e.g., adhesive bonds, pressure bonds, thermal bonds, and/or ultrasonic bonds applied after the substrate is formed) and combinations thereof. Surface modifications may be formed after the initial formation of the substrate itself, thereby forming a modified substrate. In other words, thermal bonding fibers to produce a nonwoven is not considered a surface modification. Creating bonds on the nonwoven once it has been formed is considered a surface modification. Additionally, or alternatively, surface modifications 800 on different layers may work together to provide an enhanced property.

FIG. 26B is a plan view of an exemplary side member illustrating exemplary structural features. The exemplary side members may be a first elastic side member 914 or a second elastic side member, as illustrated in FIGS. 27A-27D. One or more structural features 801 may be formed on the laminate after initial bonding of the layers. Structural features 801 may comprise embossing 802, cuts 804 (e.g., apertures, perforations, slits), melted material or coatings 806, compressed material 808, plastic deformation 810 (e.g., activation stripes 812), folds 814, secondary bonds 816 (e.g., adhesive bonds, pressure bonds, thermal bonds, and/or ultrasonic bonds applied after the initial bonding of the laminate) and combinations thereof. A structural feature or a surface modification may be in the form a design element 820.

It is to be appreciated that combinations of one or more surface modifications 800 and one or more structural features 801 may be employed. It is also to be appreciated that certain cover layer substrates may be selected for their amenability to surface modifications and/or structural features. By way of nonlimiting example, a carded nonwoven may be selected if mechanically activating the substrate and/or laminate. Lower modulus materials, such as polyethylene-based materials, may be more suitable for modification through laser energy.

### Dimensions

A variety of measurements may be useful in defining and distinguishing absorbent articles 10. All measurements are with the absorbent article laid flat and without waistband contraction, which may require the use of clamps to prevent contraction of various elastic members. “Chassis Width” is product width or the width of the chassis 52 of the absorbent article 10 at the central lateral axis 48, as shown in FIG. 1. Chassis Pitch (Length) is the product length or the length of the chassis 52 of the absorbent article 10 at the central longitudinal axis 50, as shown in FIG. 1. “Wingspan” is a width 13 between the innermost edge of one fully-extended fastener 46 to the innermost edge of an opposing fully-extended fastener 46 when the absorbent article 10 and the back ears 42 are laid flat, as shown in FIG. 1. Tables 1 and 2 provide examples of measurements of various brands of diapers designed for babies of specific weights.

TABLE 1

| | Example Sub-Brand 1 | | | | | | | |
|---|---|---|---|---|---|---|---|---|
| | 0 | 1 | 2 | 3 | 4 | 5 | 6 | 7 |
| Wingspan (mm) | 247 | 295 | 325 | 325 | 325 | 346 | 346 | 346 |
| Chassis Width (mm) | 150 | 195 | 195 | 195 | 195 | 210 | 210 | 210 |
| Chassis Pitch (mm) | 330 | 390 | 410 | 432 | 478 | 505 | 525 | 557 |
| Baby Weight Rg | less than 10 lbs | 8-14 lbs | 12-18 lbs | 16-28 lbs | 22-37 lbs | up to 27 | up to 35 | Up to 41 |

TABLE 2

| | Example Sub-brand 2 | | | | | | | |
|---|---|---|---|---|---|---|---|---|
| | 0 | 1 | 2 | 3 | 4 | 5 | 6 | 7 |
| Wingspan (mm) | 259 | 295 | 325 | 325 | 347 | 359 | 359 | — |
| Chassis Width (mm) | 143 | 195 | 195 | 195 | 195 | 225 | 225 | — |
| Chassis Pitch (mm) | 303 | 356 | 387 | 431 | 471 | 500 | 526 | — |
| Baby Weight Rg | less than 10 lbs | 8-14 lbs | 12-18 lbs | 16-28 lbs | 22-37 lbs | up to 27 | up to 35 | Up to 41 |

According to various embodiments, chassis width may be in a range of from 200 to 400 mm, or from 247 to 346 mm, or from 247 to 295 mm, or from 247 to 346 mm, or from 295 to 325 mm, or from 295 to 346 mm, or from 325 to 346 m, specifically reciting all 1 mm increments therebetween.

According to various embodiments, the wingspan of an absorbent article may be 200 to 400, or 247 mm to 359 mm, or 247 mm to 259 mm, or 247 mm to 295 mm, or 247 mm to 325 mm, or 247 mm to 346 mm, or 247 mm to 347 mm, or 247 mm to 359 mm, or 259 mm to 295 mm, or 259 mm to 325 mm, or 259 mm to 346 mm, or 259 mm to 347 mm, or 259 mm to 359 mm, or 295 mm to 325 mm, or 295 mm to 346 mm, or 295 mm to 347 mm, or 295 mm to 359 mm, or 325 mm to 346 mm, or 325 mm to 347 mm, or 325 mm to 359 mm, or 346 mm to 347 mm, or 346 mm to 359 mm, or 347 mm to 359 mm, specifically reciting all 1 mm increments therebetween.

According to various embodiments, the chassis width of an absorbent article may be 100 to 300 mm, or 143 mm to 225 mm, or 143 mm to 150 mm, or 143 mm to 195 mm, or 143 mm to 210 mm, or 143 mm to 225 mm, or 150 mm to 195 mm, or 150 mm to 210 mm, or 150 mm to 225 mm, or 195 mm to 210 mm, or 195 mm to 225 mm, or 210 mm to 225 mm, specifically reciting all 1 mm increments therebetween.

According to various embodiments, the chassis width of an absorbent article may be 250 to 650 mm, or 303 mm to 557 mm, or 303 mm to 330 mm, or 303 mm to 356 mm, or 303 mm to 387 mm, or 303 mm to 390 mm, or 303 mm to 410 mm, or 303 mm to 431 mm, or 303 mm to 432 mm, or 303 mm to 471 mm, or 303 mm to 478 mm, or 303 mm to 500 mm, or 303 mm to 505 mm, or 303 mm to 525 mm, or 303 mm to 526 mm, or 303 mm to 557 mm, or 330 mm to 356 mm, or 330 mm to 387 mm, or 330 mm to 390 mm, or 330 mm to 410 mm, or 330 mm to 431 mm, or 330 mm to 432 mm, or 330 mm to 471 mm, or 330 mm to 478 mm, or 330 mm to 500 mm, or 330 mm to 505 mm, or 330 mm to 525 mm, or 330 mm to 526 mm, or 330 mm to 557 mm, or 356 mm to 387 mm, or 356 mm to 390 mm, or 356 mm to 410 mm, or 356 mm to 431 mm, or 356 mm to 432 mm, or 356 mm to 471 mm, or 356 mm to 478 mm, or 356 mm to 500 mm, or 356 mm to 505 mm, or 356 mm to 525 mm, or 356 mm to 526 mm, or 356 mm to 557 mm, or 387 mm to 390 mm, or 387 mm to 410 mm, or 387 mm to 431 mm, or 387 mm to 432 mm, or 387 mm to 471 mm, or 387 mm to 478 mm, or 387 mm to 500 mm, or 387 mm to 505 mm, or 387 mm to 525 mm, or 387 mm to 526 mm, or 387 mm to 557 mm, or 390 mm to 410 mm, or 390 mm to 431 mm, or 390 mm to 432 mm, or 390 mm to 471 mm, or 390 mm to 478 mm, or 390 mm to 500 mm, or 390 mm to 505 mm, or 390 mm to 525 mm, or 390 mm to 526 mm, or 390 mm to 557 mm, or 410 mm to 431 mm, or 410 mm to 432 mm, or 410 mm to 471 mm, or 410 mm to 478 mm, or 410 mm to 500 mm, or 410 mm to 505 mm, or 410 mm to 525 mm, or 410 mm to 526 mm, or 410 mm to 557 mm, or 431 mm to 432 mm, or 431 mm to 471 mm, or 431 mm to 478 mm, or 431 mm to 500 mm, or 431 mm to 505 mm, or 431 mm to 525 mm, or 431 mm to 526 mm, or 431 mm to 557 mm, or 432 mm to 471 mm, or 432 mm to 478 mm, or 432 mm to 500 mm, or 432 mm to 505 mm, or 432 mm to 525 mm, or 432 mm to 526 mm, or 432 mm to 557 mm, or 471 mm to 478 mm, or 471 mm to 500 mm, or 471 mm to 505 mm, or 471 mm to 525 mm, or 471 mm to 526 mm, or 471 mm to 557 mm, or 478 mm to 500 mm, or 478 mm to 505 mm, or 478 mm to 525 mm, or 478 mm to 526 mm, or 478 mm to 557 mm, or 500 mm to 505 mm, or 500 mm to 525 mm, or 500 mm to 526 mm, or 500 mm to 557 mm, or 505 mm to 525 mm, or 505 mm to 526 mm, or 505 mm to 557 mm, or 525 mm to 526 mm, or 525 mm to 557 mm, or 526 mm to 557 mm, specifically reciting all 1 mm increments therebetween.

### Packages

The absorbent articles of the present disclosure may be placed into packages. The packages may comprise polymeric films and/or other materials. Graphics and/or indicia relating to properties of the absorbent articles may be formed on, printed on, positioned on, and/or placed on outer portions of the packages. Each package may comprise a plurality of absorbent articles. The absorbent articles may be packed under compression so as to reduce the size of the packages, while still providing an adequate amount of absorbent articles per package. By packaging the absorbent articles under compression, caregivers can easily handle and store the packages, while also providing distribution savings to manufacturers owing to the size of the packages.

### Arrays

“Array” means a display of packages comprising disposable absorbent articles of different article constructions (e.g., different elastomeric materials [compositionally and/or structurally] in the side panels, side flaps and/or belts flaps, different graphic elements, different product structures, fasteners or lack thereof). The packages may have the same brand and/or sub-brand and/or the same trademark registration and/or having been manufactured by or for a common manufacturer and the packages may be available at a common point of sale (e.g. oriented in proximity to each other in a given area of a retail store). An array is marketed as a line-up of products normally having like packaging elements (e.g., packaging material type, film, paper, dominant color, design theme, etc.) that convey to consumers that the different individual packages are part of a larger line-up. Arrays often have the same brand, for example, “Huggies,” and same sub-brand, for example, “Pull-Ups.” A different product in the array may have the same brand “Huggies” and the sub-brand “Little Movers.” The differences between the “Pull-Ups” product of the array and the “Little Movers” product in the array may include product form, application style, different fastening designs or other structural elements intended to address the differences in physiological or psychological development. Furthermore, the packaging is distinctly different in that “Pull-Ups” is packaged in a predominately blue or pink film bag and “Little Movers” is packaged in a predominately red film bag.

Further regarding “Arrays,” as another example an array may be formed by different products having different product forms manufactured by the same manufacturer, for example, “Kimberly-Clark”, and bearing a common trademark registration for example, one product may have the brand name “Huggies,” and sub-brand, for example, “Pull-Ups.” A different product in the array may have a brand/sub-brand “Good Nites” and both are registered trademarks of The Kimberly-Clark Corporation and/or are manufactured by Kimberly-Clark. Arrays also often have the same trademarks, including trademarks of the brand, sub-brand, and/or features and/or benefits across the line-up. “On-line Array” means an “Array” manufactured and/or distributed by a common on-line source.

As shown in FIGS. 27A, 27B, 27C, and 27D, an array 900 of absorbent articles 10, may comprise a plurality of absorbent articles 10, such as, for example, a first absorbent article 912 comprising a first elastic side member 914; and a second absorbent article 922 comprising a second elastic side member 924. The first absorbent article 912 and second absorbent article 922 may be produced by the same manufacturer and/or sold under the same brand name. It is to be appreciated that the absorbent articles 10 are shown in a fastened position or in the form of a pant only to simplify the illustration; the articles 10 may be in any configuration, such as folded, unfastened, and/or laid flat. The array may also be positioned within a package.

Each of the first and second elastic side members 914, 924 may comprise any version of a stretch laminate 90 described herein. For example, as shown in FIGS. 12A-12F, each of the first and second elastic side members 914, 924 may comprise at least a first cover layer 100, comprising a nonwoven material; an elastomeric film layer 300 in a face-to-face relationship with the first cover layer 100; and a plurality of ultrasonic bonds 400 joining the first cover layer 100 and the elastomeric film layer 300 to form an ultrasonically bonded stretch laminate 90.

Various elements of the absorbent articles 10 described herein may comprise a stretch laminate. For example, elastic side members, including but not limited to the belts 54 and 56 and/or the ears 42, 47 may comprise a stretch laminate 90. It is to be appreciated that the specific elements identified in FIGS. 27A to 27C are merely exemplary. As illustrated in FIG. 27A, the first absorbent article 912 may be a taped diaper and the elastic side member 916 may be a front ear or a back ear. The second absorbent article 922 may be a taped diaper and the elastic side member 926 is a front ear or a back ear. As illustrated in FIG. 27B, the first absorbent article 912 may be a pant diaper and the elastic side member 916 may be a side panel. The second absorbent article 922 may be a pant diaper and the elastic side member 926 is a side panel. As illustrated in FIG. 27A, the first absorbent article 912 may be a taped diaper and the elastic side member 916 may be a front ear or a back ear. The second absorbent article 922 may be a pant diaper and the elastic side member 926 is a side panel. It is to be appreciated that the array 900 may comprise any number of absorbent articles having the same or different configurations and properties.

Still referring to FIGS. 27A to 27C, the ultrasonically bonded stretch laminate 90 in the first absorbent article 912 may be different than the ultrasonically bonded stretch laminate 90 in the second absorbent article 914 by two or more features selected from: ultrasonic bond shape, ultrasonic bond pattern, presence of frangible bonds, elastic film basis weight, first nonwoven primary bond pattern, and first nonwoven basis weight. In other words, the first absorbent article 912 may have a first plurality of features 916 and the second absorbent article 922 may have a second plurality of features 926. The first plurality of features 916 may comprise two or more features that the second plurality of features 926 does not. Additionally or alternatively, the first plurality of features 916 may comprise two or more features having different properties than the same or similar features of the second plurality of features 926.

FIG. 27D is identical to FIG. 27B with the exception that the first elastic side member 914 and the second elastic side member 924 are discrete. “Discrete” means that the side members 914, 924 are separate components that are attached to the respective absorbent articles 912, 922 at attachment points 915, 925. The side members 914 may be attached by any suitable means, including via adhesive or ultrasonic bonds.

A variety of other differences and similarities by exist between the first absorbent article 912 and the second absorbent article 914 in an array 900. For example, at least one dimension 918 of the first absorbent article 912 may be different than at least one dimension 928 of the second absorbent article 922. The first absorbent article 912 and the second absorbent article 914 in an array 900 may be similar or may differ on the basis of any of the properties or characteristics described herein. For example, the first absorbent article or the second absorbent article may comprise an elastic waist feature in a front waist region or back waist region thereof. The first absorbent article or the second absorbent article may comprise an acquisition layer between a topsheet and an absorbent core. A portion of the acquisition layer may overlap a portion of the elastic waist feature. The first absorbent article or the second absorbent article may comprises a core bag between a topsheet and a backsheet. A portion of the core bag may overlaps a portion of the elastic waist feature. The elastic waist feature may comprise an elastic strand. The first absorbent article or the second absorbent article may comprise an absorbent core comprising an absorbent material, and the absorbent material may comprises at least 80% superabsorbent polymers by weight of the absorbent material. The absorbent material may define a channel therein.

Similarly, the first absorbent article 912 and the second absorbent article 914 in an array 900 may be similar or may differ on the basis of properties of the stretch laminate and the components made therefrom. For example, the elastic film in the first or second elastic side members may have activation stripes. The elastic film in the first or second elastic side members may have a skin on a surface thereof. A portion of the skin may have a plurality of wrinkles and the wrinkles have furrows. The ultrasonically bonded laminate may comprise apertures, slits, three-dimensional protrusions, or tinting. The first nonwoven material or the elastic film may comprise a bio-sourced resin or a recycled resin. The ultrasonic bond pattern may form one or more closed cells. The ultrasonic bond pattern may be free of one of more closed cells. At least some of the ultrasonic bonds may be frangible in an elastic region of the elastic side member. One or more of the ultrasonic bonds of the ultrasonic bond pattern may be visible when viewing a garment-facing surface of the ultrasonically bonded laminate.

The elastic side member of the first absorbent article may have a different extension than the elastic side member of the second absorbent article, according to the Tensile Test. The extension of the elastic side member of the first absorbent article may differ by 1% to 25%, or by 5% to 20%, or by 10% to 15% relative to the elastic side member of the second absorbent article according to the Tensile Test. For example, the elastic side member of the first absorbent article may have a 5% different extension than the elastic side member of the second absorbent article, according to the Tensile Test.

Any or all of the elastic side members of either the first absorbent article or the second absorbent article may have an engineering strain of 5% to 25% or of greater than about 8%, or of about 10% to 20% at about or below 2N of force when measured according to the Laminate Extension Test. For example, any or all of the elastic side members of either the first absorbent article or the second absorbent article may have an engineering strain of greater than about 8% at about or below 2N of force when measured according to the Laminate Extension Test.

### TEST METHODS

### Bond Measurement Test Method

The Bond Measurement Test Method is performed on reflected light microscopy images generated using a stereo light microscope (such as Zeiss V20 Stereoscope) and attached camera (such as the Carl Zeiss AxioCam MRc5). The image, containing at least one single repeat unit of a bond impression pattern, is acquired while the sample is fully stretched and backed with a black background. If the area of a single repeat pattern is too large for stereoscope imaging, a DSLR Camera (such as Pentax R20D), or scanner (such as Epson Perfection V750 Pro Flatbed Scanner), capable of at least a 50 micron per pixel resolution may be used to collect the image. Measurements are performed using image analysis software (such as Image Pro Plus software Version 7.0.0.591, Media Cybernetics, USA) calibrated such that distances within the image can be measured precisely to the nearest 50 microns. For purposes of this method, a bond impression is the intentional joining of two or more layers and is the deformed area caused during the bonding process (e.g., the reduced caliper at the site of bonding). Precondition samples at about 23° C.±2 C.° and about 50%±2% relative humidity for 2 hours prior to testing under the same environmental conditions.

Prior to and during image acquisition the sample is fully stretched and secured in a planar extended state. For corrugated laminates, the specimen is fully stretched when the corrugations are substantially flattened by extending the laminate while making sure that the inelastic substrates of the laminate are not plastically deformed. For laminates without corrugations, the specimen is considered fully stretched without such extension.

### Percent Bond Area

Open the image in the image analysis software and identify a single repeat unit within the pattern of bond impressions and unbonded areas. Draw a region of interest (ROI) that encompasses the single repeat unit. Calculate and record the area of the single repeat unit ROI. Next, with the irregular area tool, trace the perimeters of each individual discrete bond impression or portion thereof enclosed within the single repeat unit ROI and record their individual areas. Calculate the sum of all the individual discrete bond impression areas or portions thereof that are within the single repeat unit. Calculate Percent Bond Area as follows:

$$Percent Bond Area ( \% ) = \frac{Sum of Individual Discrete Bond Areas}{Single Repeat Unit Area} \times 100$$

Repeat this procedure for a total of 3 replicate repeat units. Calculate the arithmetic mean of the 3 values and report as Percent Bond Area to the nearest 0.1%.

Calculate the arithmetic mean of all the recorded whole, exclude any partial, individual discrete bond impression areas and report as the Discrete Bond Area to the nearest 0.01 mm².

### Bond Separation Distance

The Bond Separation Distance is defined as the shortest (minimum), straight-line distance between the perimeters of any two individual bond sites within a single closed cell unit. Using image analysis software measure and record the Bond Separation Distance for all the discrete bond sites making up the perimeter of an identified single closed cell unit. Repeat this procedure for a total of 3 replicate closed cell units. Calculate and report the arithmetic mean of the recorded values and report as the Bond Separation Distance to the nearest 0.1 mm.

### Laminate Extension Test

### 1. Back Ears

### Structures Relevant to the Back Ear

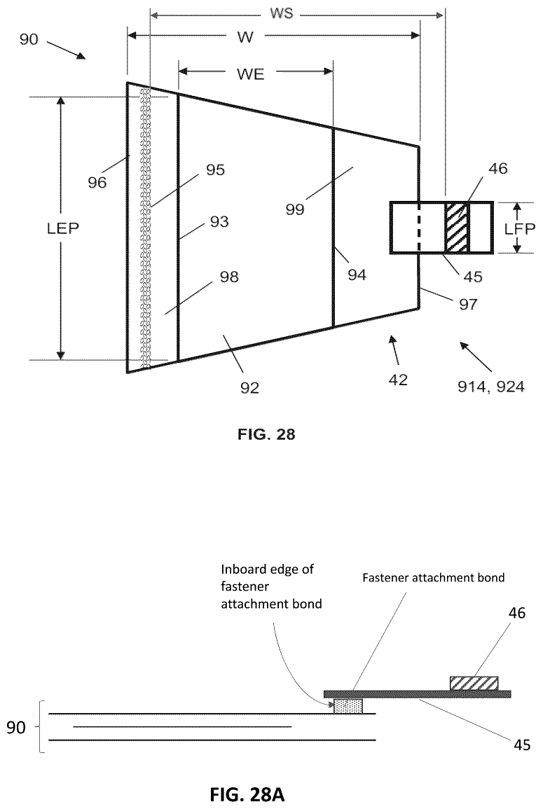

FIG. 28 is a schematic illustration showing an exemplary back ear 42 of an absorbent article, identifying features relevant for analyzing a back ear according to the Laminate Extension Test. The back ear 42 may have a total width W extending from an inboard edge 96 to an outboard edge 97. The outboard edge 97 is the free distal longitudinal edge of the ear when said ear is joined to the chassis. The inboard edge 96 is substantially opposed to the outboard edge and is joined to or overlapped with the chassis when the ear is joined to the chassis.

The back ear 42 may comprise a stretch laminate 90. The stretch laminate 90 make up all or a portion of the total width W of the back ear 42. The back ear 42 may comprise an elastic region 92. The elastic region 92 may coincide with all or a portion of the stretch laminate 90. The elastic region 92 may have a width WE, extending from an inboard edge 93 to an outboard edge 94 of the elastic region 92. The width WE of the elastic region 92 may be less than or equal to the total width W of the back ear 42. The inbounded edge 93 of the elastic region 92 may have a length LEP. The back ear 42 may further comprise a fastener 46, having a length LFP. In some embodiments, the area of the elastic region comprises at least about 20% of, or from about 30% to about 100% of the total area of the ear, reciting for said range every 5% increment therein.

The back ear 42 may further comprise one or more inelastic regions. In certain embodiments, the back ear 42 comprises a first inelastic region 98, which extends laterally outward from the inboard edge 96 and is adjacent to the elastic region 92 at an inboard edge 93 of the elastic region 92. The ear may further include a second inelastic region 99, which may extend laterally inward from the outboard edge 97 and may be adjacent to outboard edge 94 of the elastic region 92. The first and second inelastic regions may be made of the same material(s) or different materials.

Still referring to FIG. 28, a reference width WS may be identified, as the width from a junction line (as defined in the following paragraph) to an inboard edge 45 of the fastener 46. In some examples, the fastener 46 may have an irregular shape or orientation, or consist of a plurality of engaging portions; in such examples, the point at which such shape, orientation or extensible portions are closest to a longitudinal axis of an absorbent article is considered the inboard edge 45 of the fastener 46.

As used herein, the term “junction line,” with respect to a back ear comprising components that are discrete from other components of an absorbent article, which back ear is welded, bonded, adhered or otherwise attached to the absorbent article, means a longitudinal line 95, parallel with a longitudinal axis of the absorbent article, through the outboard-most point in a chassis attachment bond at which the back ear is bonded to the chassis. Note: In some examples of back ears, the back ear is bonded to the chassis to have an irregular shape or orientation; in such examples, the point at which such shape or orientation are closest to an outboard edge of the back ear will mark the location of the junction line. “Junction line,” with respect to a back ear comprising one or more components that are not discrete from, but rather, integral with, one or more components of a diaper chassis that is disposed in an opened, extended position and laid flat and horizontally, viewed from above means a line parallel to the longitudinal axis through the edge of the chassis at its narrowest point.

### Preparation of Back Ear Specimens

To prepare a back ear specimen for the Laminate Extension Test the following procedure may be employed:

1. Open a diaper.

2. If the back ear 42 is attached to an article, cut it free from the article at a location sufficiently inboard of the junction line 95 so that a tensile tester's grip can sufficiently grasp the specimen for the testing beyond the junction line 95. If the back ear 42 is an integral part of a chassis, identify the junction line 95 and mark a line on the back ear 42 coincident with the junction line 95, and cut it free from the article at a location sufficiently inboard of the junction line 95 so that a tensile tester's grip can sufficiently grasp the specimen for the testing beyond the junction line.

3. Lay the back ear 42 on a substantially flat, horizontal surface and measure width WS as described herein, with no lateral tension force applied to the back ear 42.

4. Measure length LFP as described herein to the nearest 1 mm, with a steel ruler traceable to NIST or an equivalent.

5. Mark a midpoint of LFP. The midpoint is at ½ of LFP.

### Testing Back Ears Specimens

An engineering strain and an extension of a back ear specimen is measured using a constant rate of extension tensile tester with computer interface such as MTS Alliance under Test Works 4 software (MTS Systems Corp., USA) fitted with a suitable load cell. The load cell should be selected to operate within 10% and 90% of its stated maximum load. All testing is performed in a conditioned room maintained at about 23° C.±2° C. and about 50%±2% relative humidity. Herein, width and length of the specimen are a lateral width and longitudinal length as defined herein. Precondition specimens at about 23° C.±2° C. and about 50%±2% relative humidity for 2 hours prior to testing.

1. Insert the outboard edge 97 of the back ear 42 including a fastener 46 into the upper clamp in the tester such that the clamp is centered in the tensile tester fixture and engage the clamp to grip the specimen. The clamp width is at least as wide as the length of an inboard edge of the fastener 46, and preferably is not wider than 1 inch beyond the length of an inboard edge of the fastener 46. The face of the clamp (once it grips the specimen) is aligned with the inboard edge of the fastener 46 to within 1 mm, the longitudinal midpoint of LFP is aligned with the center of the clamp, and the unclamped portion of the back ear hangs freely downward from the upper clamp.

2. Insert the inboard edge 96 of the back ear 42 into the lower clamp in the tensile tester. The lower clamp width is chosen such that no portion of the back ear 42 extends beyond the width of the clamp, and preferably the lower clamp width is not wider than 1 inch beyond the length of the portion of the back ear 42 inserted into the clamp. The face of the clamp (once it grips the specimen) is aligned with the junction line 95 to within 1 mm, and the specimen is oriented such that if a lateral line perpendicular to a longitudinal axis of the diaper having the back ear 42 were drawn from the midpoint of LFP, it would extend vertically and align with the center of the fixture holding the lower clamp.

3. Extend the jaws of the tensile tester such that the distance between the face of the upper clamp and face of the lower clamp is equal to WS. Set gage length equal to WS.

4. Zero the crosshead location and load, and engage the lower clamp to grip the specimen.

5. Set the tensile tester to extend the specimen at a rate of 254 mm/minute and collect data at a frequency of at least 100 hz.

6. Initiate the test such that the tensile tester's clamp extends the specimen at the defined rate and data including extension and load is collected into a data file.

7. Measure an extension, a distance extended from a zero-point, under load at 2N, and determine an engineering strain under load at 2N, calculated as:

100%×[extension at 2N load/WS (at no lateral tension load)].

8. In like fashion, a total of three (3) replicate samples are tested for each test product to be evaluated. Report the extension as the mean of the replicates to the nearest 0.1 cm, and engineering strain as the mean of the replicates to the nearest 0.1 unit.

### 2. Side Members

### Preparation of Side Member Specimens

To prepare belt side member specimens for the Laminate Extension:

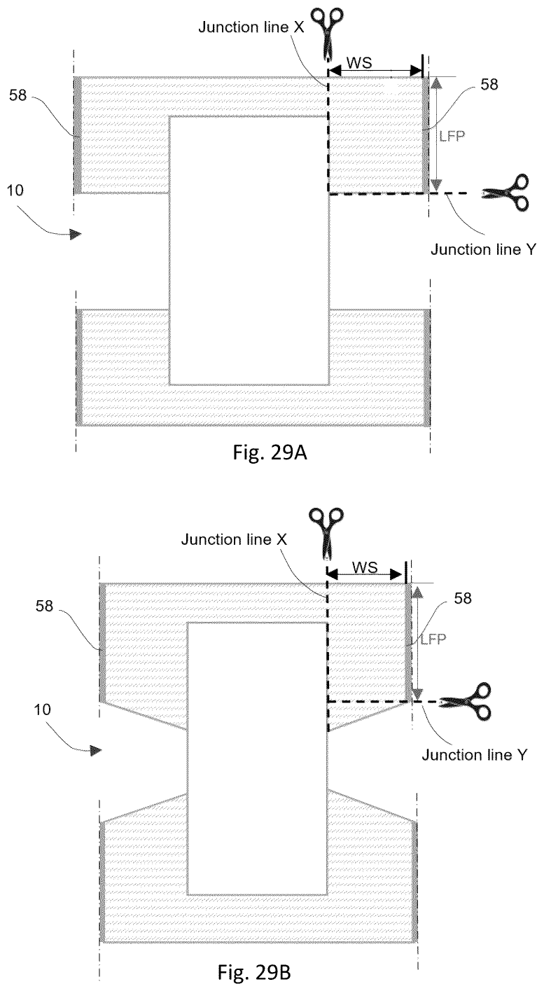

1. Open the pant 10 by separating the belt side members where they attach to each other, such as the side seam 58 illustrated in FIG. 4. This is performed by cutting through the fused or permanently bonded side seam region 58 with an exacto knife, scissors or equivalent, such as illustrated in FIGS. 29A and 29B. Cut though the side seam such that enough of the side seam remains available for gripping the sample onto the clamps of the tensile tester. In the case of refastenable pants, the side members are not permanently fused or bonded to each other along the lateral sides of the article but instead are attached to each other by releasable fasteners (usually mechanical fastening systems). In these cases, open the pant by carefully disengaging or separating the fasteners that releasably connect the side members to each other along the lateral sides of the article.

2. Cut the belt side member free from the article at a lateral location at the junction line X so that a grip on the tensile tester can sufficiently grasp the specimen for the testing at the junction line X. The junction line X is a line parallel to the longitudinal axis of the article that is located at the outboard edge of the absorbent structure. If the absorbent structure is irregularly shaped, the outboard edge is considered to be the farthest lateral outboard point of the irregularly shaped absorbent structure. Also, cut the belt side member along junction line Y. The junction line Y is a line parallel to the lateral axis of the article that is located at the longitudinal inboard edge of the side seam such as illustrated in FIGS. 29A and 29B. The junction line Y may not be a necessary cut if the belt is rectangularly shaped and no longer than the side seam.

2a. In the event, that the pant article 10 contains discrete side members attached to the chassis instead of belts, cut the side member specimen at junction line 95 (consistent with the sampling procedure for Taped diaper back ears). Also, cut the discrete side member along the junction line Y as described herein and continue to step 3.

3. Lay the side member on a substantially flat, horizontal surface and measure width WS with no lateral tension force applied it. WS is distance between the junction line X and the lateral inboard edge of previously separated side seam region 58 as illustrated in FIGS. 29A and 29B.

4. Measure length LFP to the nearest 1 mm, with a steel ruler traceable to NIST or an equivalent. LFP is the longitudinal length of the side member specimen at the outboard cut edge.

5. Mark a midpoint of LFP. The midpoint is half of LFP.

### Testing for Side Member Specimens

### Testing of Side Members

An engineering strain and an extension of a side member specimen is measured using a constant rate of extension tensile tester with computer interface such as MTS Alliance under Test Works 4 software (MTS Systems Corp., USA) fitted with a suitable load cell. The load cell should be selected to operate within 10% and 90% of its stated maximum load. All testing is performed in a conditioned room maintained at about 23° C.±2° C. and about 50%±2% relative humidity. Herein, width and length of the specimen are a lateral width and longitudinal length as defined herein. Precondition specimens at about 23° C.±2° C. and about 50%±2% relative humidity for 2 hours prior to testing.

1. Insert the outboard edge of the side member into the upper clamp in the tester such that the clamp is centered in the tensile tester fixture and engage the clamp to grip the specimen. Grip the specimen by the side seam region 58 which comprises previously separated permanent side seam attachment bonds or releasable fasteners in the case of refastenable pants. The clamp width is at least as wide as the length of the outboard edge, and preferably is not wider than 1 inch beyond the length of the outboard edge of the specimen. The clamp is set to grip in a location aligned with the outboard edge of the side member within 1 mm, the longitudinal midpoint of the outboard edge of the side member is aligned with the center of the clamp, and the unclamped portion of the side member hangs freely downward from the upper clamp.

2. Insert the inboard edge of the side member into the lower clamp in the tensile tester. The lower clamp width is chosen such that no portion of the side member extends beyond the width of the clamp, and preferably the lower clamp width is not wider than 1 inch more than the length of the portion of the side member inserted into the clamp. The clamp is set to grip in a location aligned with the junction line X to within 1 mm, and the specimen is oriented such that if a lateral line perpendicular to a longitudinal axis of the pant having the side members were drawn from the midpoint of LFP, it would extend vertically and align with the center of the fixture holding the lower clamp.

3. Extend the jaws of the tensile tester such that the clamped distance between upper and lower grip locations clamp is equal to WS. Set gage length equal to WS.

4. Zero the crosshead location and load, and engage the lower clamp to grip the specimen.

5. Set the tensile tester to extend the specimen at a rate of 254 mm/minute and collect data at a frequency of at least 100 hz.

6. Initiate the test such that the tensile tester's clamp extends the specimen at the defined rate and data including extension and load is collected into a data file.

7. Measure an extension, a distance extended from a zero-point, under load at 2N, and determine an engineering strain under load at 2N, calculated as:

100% strain=[extension at 2N load/WS (at no lateral tension load)].

8. In like fashion, a total of three (3) replicate samples are tested for each test product to be evaluated. Report the extension as the mean of the replicates to the nearest 0.1cm, engineering strain as the mean of the replicates to the nearest 0.1 unit and percent strain to the nearest 0.1%

### Tensile Test Method

The Tensile Test is described and illustrated in US Pat. Pub. No. 2018/0042786, titled Array of Absorbent Articles with Ear Portions, by Mueller et al., which is incorporated by reference in its entirety. The Tensile Test is used to measure the strength of a specimen at a relatively high strain rate that represents product application. The method uses a suitable tensile tester such as an MTS 810, available from MTS Systems Corp., Eden Prairie Minn., or equivalent, equipped with a servo-hydraulic actuator capable of speeds exceeding 5 m/s after 28 mm of travel, and approaching 6 m/s after 40 mm of travel. The tensile tester is fitted with a 50 lb. force transducer (e.g., available from Kistler North America, Amherst, N.Y. as product code 9712 B50 (50 lb)), and a signal conditioner with a dual mode amplifier (e.g., available from Kistler North America as product code 5010). Grips should be used to secure the specimens during tensile testing. The opposing grips may have the same width or different widths as specified. All testing is performed in a conditioned room maintained at about 23° C.±2° C. and about 50%±2% relative humidity. Herein, width and length of the specimen are a lateral width and longitudinal length as defined herein. Precondition specimens at about 23° C.±2° C. and about 50%±2% relative humidity for 2 hours prior to testing.

### (a) Grips

The line grips are selected to provide a well-defined gauge and avoid undue slippage. The specimen is positioned such that it has minimal slack between the grips. A portion of one or both grips may be configured to include a material that reduces the tendency of a specimen to slip, (e.g., a piece of urethane or neoprene rubber having a Shore A hardness of between 50 and 70). Six inches wide top and bottom grips are used to clamp the specimen unless specified otherwise.

(b) Tensile Test of Specimen from Absorbent Article—Back Ears

Ears are generally bonded to the chassis via thermal or adhesive or similar bonding. Ears should be separated from the chassis in a way that ears are not damaged and performance of the ear is not altered. If the chassis bond is too strong (i.e., ears will be damaged upon removal), then the portion of the chassis joined to the ear should be cut within the chassis material but without damaging the ear. Folded fastening systems (e.g., release tapes covering fastening elements) should be unfolded.

The specimen is clamped in the top grip at a first grip location, which is inboard of the fastener attachment bond, as illustrated in FIG. 28A. The grip line is kept parallel to the longitudinal centerline of the product. If the fastener attachment bond is angled, the specimen is gripped at the center of the bond region and grip line is kept parallel to the longitudinal centerline of the product at the center. The width of the top grip should be equal to the maximum length of the fastener attachment bond measured parallel to the longitudinal centerline of the article. If, at the first grip location position, the length of the specimen is the same as the maximum length of the fastener attachment bond, then any grip width greater than the specimen length at the first grip location can be used. The specimen is mounted and hung from the top grip. The opposing edge of the specimen is mounted in the bottom grip in relaxed condition. The bottom grip location is adjusted so the specimen is gripped at the outboard edge of the chassis bond. If the chassis bond is curvilinear, the specimen is gripped at the outboard edge of the outermost bond, such as indicated in FIG. 28 as the junction line 95. The bottom grip is greater than the length of the ear at the second grip location. The top and bottom grips are parallel to each other.

The specimen is tested as follows: The vertical distance (perpendicular to the grip line) from the first grip location to the second grip location is measured to 0.1 mm using ruler and is used as gage length for the test. The specimen is tested at a test speed that provides 9.1 sec⁻¹ strain rate with the gage length selected for the specimen. Test speed in mm/second is calculated by multiplying 9.1 sec⁻¹ by the gage length in mm. Before testing, 5 mm of slack is put between the grips.

Each specimen is pulled to break. During testing, one of the grips is kept stationary and the opposing grip is moved. The force and actuator displacement data generated during the test are recorded using a MOOG SmarTEST ONE STO03014-205 standalone controller, with the data acquisition frequency set at 1 kHz. The resulting load data may be expressed as load at break in Newton. The Extension (mm) at 5 N and at 10 N are also recorded. Total of five (5) specimens are run for example. The Average Load at Break and standard deviation, the Average Extension at 5N and standard deviation, and the Average Extension at 10 N and standard deviation are recorded. If, standard deviation recorded is higher than 5%, a new set of five specimens is run.

### (c) Length Ratio—Back Ears

Per the earlier steps, the grips are positioned at a first grip location and a second grip location. The ratio of the length of the specimen at the second grip position (L2) to the first grip position (L1) is Length Ratio. The respective lengths are measured to 0.1 mm accuracy using the ruler.

(d) Tensile Test of Specimen from Absorbent Article-Side Members
To prepare belt side member specimens for the Tensile Test:

1. Open the pant 10 by separating the belt side members where they attach to each other, such as the side seam 58 illustrated in FIG. 4. This is performed by cutting through the fused or permanently bonded side seam region 58 with an exacto knife, scissors or equivalent, such as illustrated in FIGS. 29A and 29B. Cut though the side seam such that enough of it remains available for gripping the sample onto the clamps of the tensile tester. In the case of refastenable pants, the side members are not permanently fused or bonded to each other along the lateral sides of the article but instead are attached to each other by releasable fasteners (usually mechanical fastening systems). In these cases, open the pant by carefully disengaging or separating the fasteners that releasably connect the side members to each other along the lateral sides of the article.

2. Cut the belt side member free from the article at a lateral location at the junction line X so that a grip on the tensile tester can sufficiently grasp the specimen for the testing at the junction line X. The junction line X is a line parallel to the longitudinal axis of the article that is located at the outboard edge of the absorbent structure. If the absorbent structure is irregularly shaped, the outerboard edge is considered to be the farthest lateral outboard point of the irregularly shaped absorbent structure. Also, cut the belt side member along junction line Y. The junction line Y is a line parallel to the lateral axis of the article that is located at the longitudinal inboard edge of the side seam such as illustrated in FIGS. 29A and 29B. The junction line Y may not be a necessary cut if the belt is rectangularly shaped and no longer than the side seam.

2a. In the event, that the pant article 10 contains discrete side members attached to the chassis instead of belts, cut the side member specimen at junction line 95 (consistent with the sampling procedure for Taped diaper back ears). Also, cut the discrete side member along the junction line Y as described herein and continue to step 3.

3. Lay the side member on a substantially flat, horizontal surface and measure width WS with no lateral tension force applied it. WS is distance between the junction line X and the lateral inboard edge of previously separated side seam region 58 as illustrated in FIGS. 29A and 29B.

4. Measure length LFP to the nearest 1 mm, with a steel ruler traceable to NIST or an equivalent. LFP is the longitudinal length of the side member specimen at the outboard cut edge.

5. Mark a midpoint of LFP. The midpoint is half of LFP.

### To Test Side Members for Tensile Testing

1. Insert the outboard edge of the side member into the upper clamp in the tester such that the clamp is centered in the tensile tester fixture and engage the clamp to grip the specimen. Grip the specimen by the side seam region 58 which comprises previously separated permanent side seam attachment bonds or releasable fasteners in the case of refastenable pants. The clamp width is at least as wide as the length of the outboard edge, and preferably is not wider than 1 inch beyond the length of the outboard edge of the specimen. The clamp is set to grip in a location aligned with the outboard edge of the side member within 1 mm, the longitudinal midpoint of the outboard edge of the side member is aligned with the center of the clamp, and the unclamped portion of the side member hangs freely downward from the upper clamp.

2. Insert the inboard edge of the side member into the lower clamp in the tensile tester and in relaxed condition. The lower clamp width is chosen such that no portion of the side member extends beyond the width of the clamp, and preferably the lower clamp width is not wider than 1 inch more than the length of the portion of the side member inserted into the clamp. The clamp is set to grip in a location aligned with the junction line X to within 1 mm, and the specimen is oriented such that if a lateral line perpendicular to a longitudinal axis of the pant having the side members were drawn from the midpoint of LFP, it would extend vertically and align with the center of the fixture holding the lower clamp.

The specimen is tested as follows: The vertical distance (perpendicular to the grip line) from the first grip location to the second grip location is measured to 0.1 mm using ruler and is used as gage length for the test. The specimen is tested at a test speed that provides 9.1 sec⁻¹ strain rate with the gage length selected for the specimen. Test speed in mm/second is calculated by multiplying 9.1 sec⁻¹ by the gage length in mm. Before testing, 5 mm of slack is put between the grips.

Each specimen is pulled to break. During testing, one of the grips is kept stationary and the opposing grip is moved. The force and actuator displacement data generated during the test are recorded using a MOOG SmarTEST ONE STO03014-205 standalone controller, with the data acquisition frequency set at 1 kHz. The resulting load data may be expressed as load at break in Newton. The Extension (mm) at 5 N and at 10 N are also recorded. Total of five (5) specimens are run for example. The Average Load at Break and standard deviation, the Average Extension at 5N and standard deviation, and the Average Extension at 10 N and standard deviation are recorded. If, standard deviation recorded is higher than 5%, a new set of five specimens is run.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An array of absorbent articles, the array comprising:
a first absorbent article comprising a first elastic side member; and
a second absorbent article comprising a second elastic side member;
wherein each of the first and second elastic side members comprises:
a first nonwoven material;
an elastic film in a face-to-face relationship with the first nonwoven material; and
a plurality of ultrasonic bonds joining the first nonwoven material and the elastic film to form an ultrasonically bonded laminate;
wherein the ultrasonically bonded laminate in the first absorbent article is different than the ultrasonically bonded laminate in the second absorbent article by two or more features selected from: ultrasonic bond shape, ultrasonic bond pattern, presence of frangible bonds, elastic film basis weight, first nonwoven primary bond pattern, and first nonwoven basis weight;
wherein at least one dimension of the first absorbent article is different than at least one dimension of the second absorbent article; and
wherein the first and second absorbent articles are produced by the same manufacturer and/or sold under the same brand name.

2. The absorbent article of claim 1, wherein each of the first and second elastic side members comprises a second nonwoven material in a face-to-face relationship with the elastic film, wherein the elastic film is positioned intermediate the first nonwoven materials and the second nonwoven materials, and wherein the plurality of ultrasonic bonds join the first nonwoven material, the elastic film, and the second nonwoven material to form the ultrasonically bonded laminate.

3. The absorbent article of claim 1, wherein the ultrasonically bonded laminate in the first absorbent article is different than the ultrasonically bonded laminate in the second absorbent article by ultrasonic bond density per unit area.

4. The absorbent article of claim 2, wherein the first nonwoven material and the second nonwoven material of the first elastic side member each have only a single layer, and wherein the first nonwoven material or the second nonwoven material of the second elastic side member has two layers in a portion thereof.

5. The absorbent article of claim 1, wherein the elastic film in the first and second elastic side members is preactivated prior to being ultrasonically bonded to the first nonwoven material, and wherein the elastic film is joined to the first nonwoven material when the elastic film is in a stretched state.

6. The absorbent article of claim 1, wherein the elastic film in the first and second elastic side members has activation stripes.

7. The absorbent article of claim 1, wherein the elastic film in the first and second elastic side members has a skin on a surface thereof, and wherein a portion of the skin has a plurality of wrinkles and the wrinkles have furrows.

8. The absorbent article of claim 1, wherein the first absorbent article or the second absorbent article comprises an elastic waist feature in a front waist region or back waist region thereof.

9. The absorbent article of claim 8, wherein the first absorbent article or the second absorbent article comprises an acquisition layer between a topsheet and an absorbent core, and wherein a portion of the acquisition layer overlaps a portion of the elastic waist feature.

10. The absorbent article of claim 8, wherein the first absorbent article or the second absorbent article comprises a core bag between a topsheet and a backsheet, and wherein a portion of the core bag overlaps a portion of the elastic waist feature.

11. The absorbent article of claim 8, wherein the elastic waist feature comprises an elastic strand.

12. The absorbent article of claim 1, wherein the ultrasonically bonded laminate comprises apertures, slits, three-dimensional protrusions, or tinting.

13. The absorbent article of claim 1, wherein the first nonwoven material or the elastic film comprise a bio-sourced resin or a recycled resin.

14. The absorbent article of claim 1, wherein the ultrasonic bond pattern forms one or more closed cells.

15. The absorbent article of claim 1, wherein the ultrasonic bond pattern is free of one of more closed cells.

16. The absorbent article of claim 1, wherein at least some of the ultrasonic bonds are frangible in an elastic region of the elastic side member.

17. The absorbent article of claim 1, wherein one or more of the ultrasonic bonds of the ultrasonic bond pattern are visible when viewing a garment-facing surface of the ultrasonically bonded laminate.

18. The absorbent article of claim 1, wherein the first absorbent article is a taped diaper, wherein the elastic side member is a front ear or a back ear, wherein the second absorbent article is a taped diaper, and wherein the elastic side member is a front ear or a back ear.

19. The absorbent article of claim 1, wherein the first absorbent article or the second absorbent article comprises an absorbent core comprising an absorbent material, and wherein the absorbent material comprises at least 80% superabsorbent polymers by weight of the absorbent material.

20. The absorbent article of claim 1, wherein the elastic side member of the first absorbent article has a 5% different extension than the elastic side member of the second absorbent article, according to the Tensile Test, and wherein the elastic side member of the first absorbent article has an engineering strain greater than about 8% at about or below 2N of force when measured according to the Laminate Extension Test.
